(19) United States
(12) Patent Application Publication
(10) Pub. No.: US 2007/0105783 A1
(43) Pub. Date: May 10, 2007

(54) Process for the manufacture of peptide facilitators of reverse cholesterol transport

(76) Inventors: Jagadish C. Sircar, San Diego, CA (US); James J. Mencel, North Wales, PA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/590,187

(22) Filed: Oct. 31, 2006

Related U.S. Application Data
(60) Provisional application No. 60/733,352, filed on Nov. 4, 2005.

(51) Int. Cl. A61K 38/05; C07K 5/06

(52) U.S. Cl. 514/18; 530/331

(57) ABSTRACT

The embodiments provide solution phase processes for making amino acid-derived compositions that enhance reverse cholesterol transport in mammals. The compositions are suitable for oral delivery and useful in the treatment and/or prevention of disease conditions associated with hypercholesterolemia.

### RELATED APPLICATIONS

This application claims priority to U.S. provisional application No. 60/733,352, filed Nov. 4, 2005, which is incorporated herein by reference.

### FIELD OF THE INVENTION

1. Field of the Invention

The present invention relates to methods for synthesizing peptide, peptide derivatives and small molecule mediators of reverse cholesterol transport (RCT) for treating hypercholesterolemia and associated cardiovascular diseases.

2. Description of the Related Art

Attempts have been made to prepare peptides that mimic ApoA-I. Since the activity of ApoA-I has been attributed to the presence of multiple repeats of a unique secondary structural feature in the protein—a class A amphipathic α-helix (Segrest, 1974, FEBS Lett. 38:247-253; Segrest et al., 1990, PROTEINS: Structure, Function and Genetics 8:103-117), most efforts to design peptides that mimic the activity of ApoA-I have focused on designing peptides that form class A-type amphipathic α-helices (See e.g., Background discussions in U.S. Pat. Nos. 6,376,464 and 6,506,799; incorporated herein in their entirety by reference thereto). Peptidic mediators of RCT, methods for their preparation, their purification, their pharmaceutical formulation, their use in methods of treatment, and analysis of their structure and function and evaluation for their potential clinical efficacy using various assays, are described in International Patent Application, Publication-No. WO 2004/094471, which is incorporated herein in its entirety by reference thereto.

### SUMMARY OF THE INVENTION

There is a need for the development of stable molecules that facilitate RCT and that are relatively simple and cost-effective to produce. Preferably, candidate molecules would mediate both indirect and direct RCT. Such molecules would have broad functional spectra. Active peptides have been synthesized using the solution phase peptide synthetic techniques disclosed herein.

Embodiments provide a method of preparing a compound having the formula: 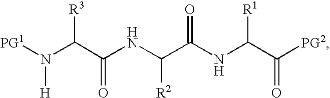
or a pharmaceutically acceptable salt thereof,
wherein R¹ is a basic side chain and R³ is an acidic side chain or
R¹ is an acidic side chain and R³ is a basic side chain;
R¹ and R³ can independently be a protected or unprotected side chain;
R² is a hydrophobic side chain;
PG¹ and PG² are protecting or capping groups;
the method comprising coupling a compound of the formula: 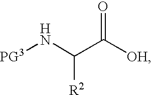
   wherein PG³ is a protecting group, and a compound of the formula: 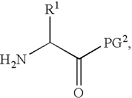
   or a mineral acid salt or organic acid salt thereof, thereby forming a compound having the formula: 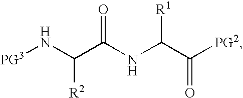
   or a mineral acid salt or an organic acid salt thereof, deprotecting the compound of the formula: 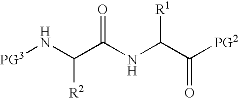
   or a mineral acid salt or an organic acid salt thereof by removing PG³, thereby forming a compound having the
   formula: 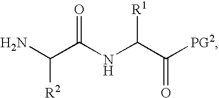
   or a mineral acid salt or an organic acid salt thereof, coupling the compound of the formula: 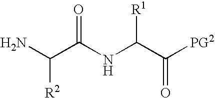
   or a mineral acid salt or an organic acid salt thereof and a compound of the formula: 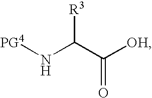
   wherein PG⁴ is a protecting group, thereby forming a compound of the formula: 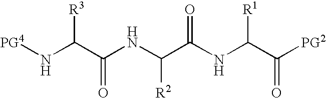
   or a mineral acid salt or an organic acid salt thereof, deprotecting the compound of the formula: 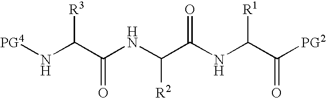
   or a mineral acid salt or an organic acid salt thereof by removing PG⁴, thereby forming a compound having the formula: 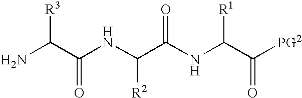
   or a mineral acid salt or an organic acid salt thereof; and
   protecting the compound of the formula: 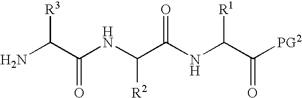
   or a mineral acid salt or an organic acid salt thereof at the terminal amino end, thereby forming a compound having the formula: 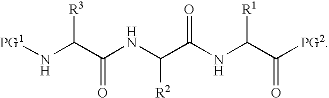
   or a mineral acid salt or an organic acid salt thereof.

In a further embodiment, PG¹ and/or PG² may optionally be removed. In some preferred embodiments, the mineral acid salt or organic acid salt is converted to a pharmaceutially acceptable salt.

In another embodiment, when the compound is obtained in a free (non-salt) form, the compound may optionally be converted to a pharmaceutically acceptable salt form; and when the compound is obtained in a salt form, it may optionally be converted to a different pharmaceutically acceptable salt form.

In the methods and compounds described herein, each amino acid is independently L or D. In another embodiment, all of the amino acids are L, and in another embodiment, all of the amino acids are D.

In preferred embodiments, one or more of the coupling steps is facilitated with a coupling agent. Preferably, the coupling agent is pivaloyl chloride or TBTU.

In preferred embodiments, PG³ and PG⁴ are benzyloxycarbonyl groups.

In preferred embodiments, one or more of the deprotecting step is performed by hydrogenolysis. Most preferably, the deprotecting step is performed with Pd(OH)₂ and hydrogen.

In preferred embodiments, PG² is an amino group.

In preferred embodiments, PG¹ is a group R^{x}—CO— wherein R^{x} is methyl, phenyl-CH₂—, di-tert-butyl-4-hydroxy-phenyl, naphthyl, substituted naphthyl, 9-fluorenylmethoxy-, biphenyl, substituted phenyl, substituted heterocycles, alkyl, aryl, substituted aryl, cycloalkyl, fused cycloalkyl, saturated heteroaryl, or substituted saturated heteroaryl. More preferably, PG¹ is an acetyl group. Preferably, the acetylation step is performed with acetic anhydride.

In some embodiments, the method includes removing PG¹ of the compound having the formula:

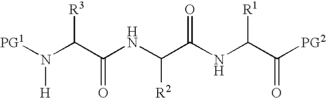

In some embodiments, the method includes removing PG² of the compound having the formula:

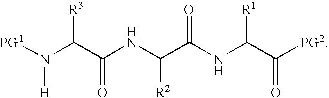

Embodiments provide a method of preparing a compound having the formula: 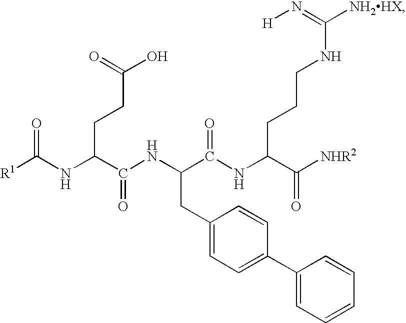
wherein R¹ is CH₃ or unsubstituted or substituted alkyl or an unsubstituted or substituted aryl and R2 is preferably H or an unsubstituted or substituted alkyl or an unsubstituted or substituted aryl; and wherein HX is a mineral acid salt, organic acid salt or a pharmaceutically acceptable salt;
the method comprising coupling a compound of the formula: 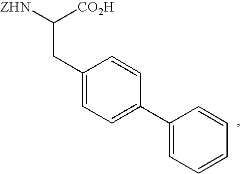
wherein Z is a benzyloxycarbonyl group, and a compound of the formula: 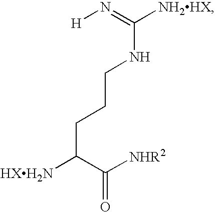
thereby forming a compound of the formula: 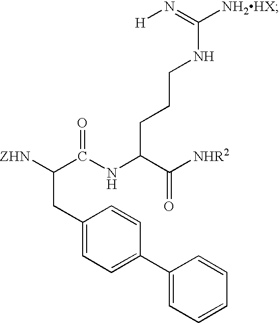
deprotecting the compound of the formula: 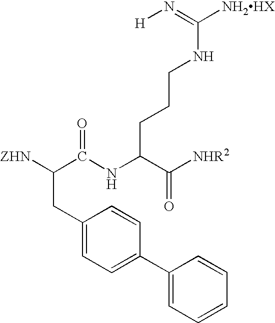
by removing the benzyloxycarbonyl group at the amino end, thereby forming a compound of the formula 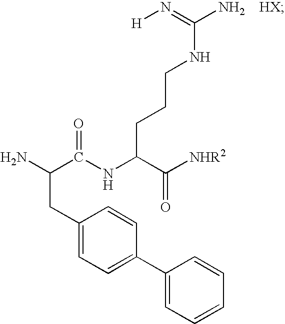
coupling the compound of the formula: 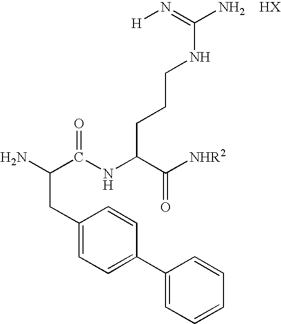
and a compound of the formula: 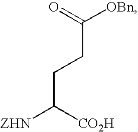
wherein Z is a benzyloxycarbonyl group and Bn is a benzyl group, thereby forming a compound of the formula: 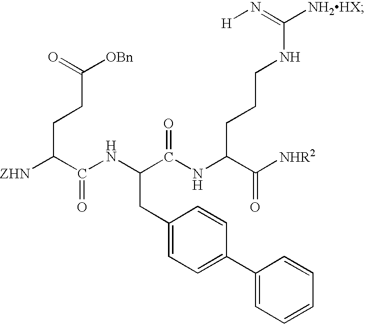
deprotecting the compound of the formula: 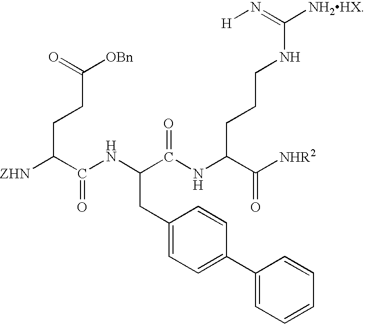
by removing the benzyloxycarbonyl group and the benzyl group, thereby forming a compound of
the formula: 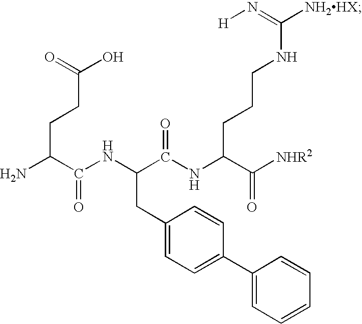
and
acylating the compound of the formula: 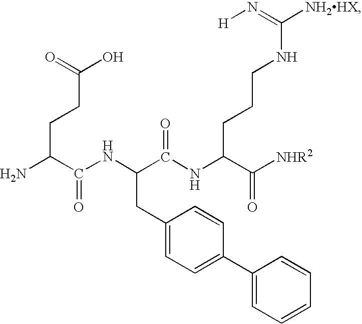
thereby forming a compound of the formula:

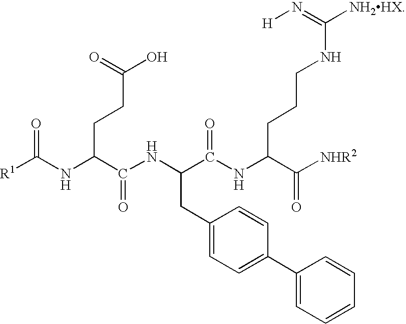

In preferred embodiments, each amino acid is independently L or D. In some embodiments, all of the amino acids are L. In some preferred embodiments, all of the amino acids are D. In some preferred embodiments, HX is HCl. In preferred embodiments, R¹ is unsubstituted alkyl which is —(CH₂)ₙ—CH₃, wherein n is 0-5. In some preferred embodiments R² is hydrogen.

Preferably, one or more of the coupling steps are facilitated with a coupling agent. In preferred embodiments, the coupling agent is pivaloyl chloride or TBTU.

Preferably, one or more of the deprotecting steps is performed by hydrogenolysis. More preferably, one or more of the deprotecting steps is performed with Pd(OH)₂ and hydrogen.

In a preferred embodiment, the acylation step is performed with acetic anhydride.

In preferred embodiments, one or more intermediates are isolated by washing an organic phase containing the intermediate with a saturated salt solution; and precipitating the intermediate from the organic phase. Preferably, the precipitation occurs by distilling the organic phase until the intermediate crystallizes out of the organic phase. For example, in the preparation of Z-(D)BIP-(D)Arg-NH₂.HCl, after the coupling reaction is complete the organic phase is separated and preferably washed first with a mixture of brine and NMM, then with a mixture of brine and aqueous hydrochloric acid, and then with brine. The organic phase is then diluted with THF and MTBE and the mixture is distilled under vacuum, with the addition of further MTBE and further distillation, whereby Z-(D)BIP-(D)Arg-NH₂.HCl crystallizes from the mixture. In preferred embodiments, the final compound is recrystallized from acetic acid or a mixture of acetic acid and ethyl acetate. Embodiments include compounds produced by the described methods. In preferred embodiments of the final compound, HX is HCl, R is hydrogen and all of the amino acids are D.

Embodiments provide a compound having the formula: 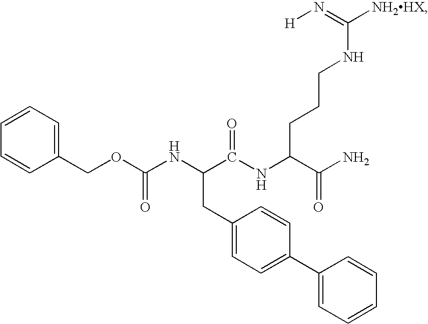
wherein HX is a mineral acid salt or organic acid salt.

Embodiments provide a compound having the formula: 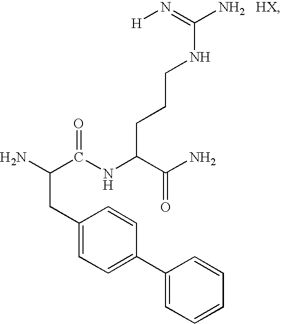
wherein HX is a mineral acid salt or organic acid salt.

Embodiments provide a compound having the formula: 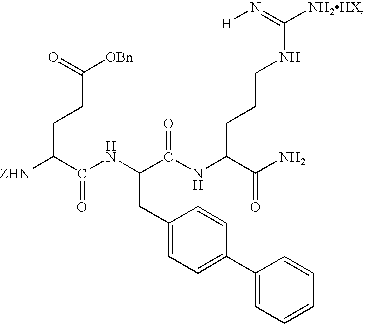
wherein HX is a mineral acid salt or organic acid salt.

Embodiments provide a compound having the formula: 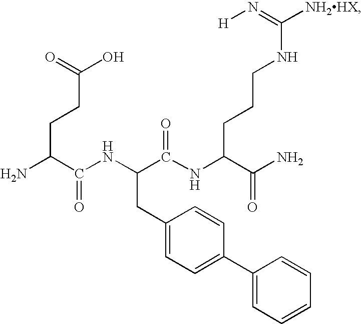
wherein HX is a mineral acid salt or organic acid salt.

Embodiments provide a compound having the formula: 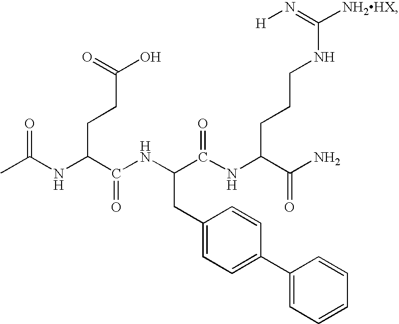
wherein HX is a mineral acid salt or organic acid salt.

Embodiments provide a compound having the formula: 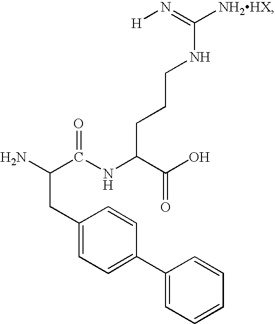
wherein HX is a mineral acid salt or organic acid salt.

Embodiments provide a compound having the formula: 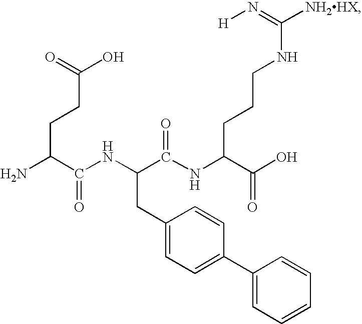
wherein HX is a mineral acid salt or organic acid salt.

Embodiments provide a compound having the formula: 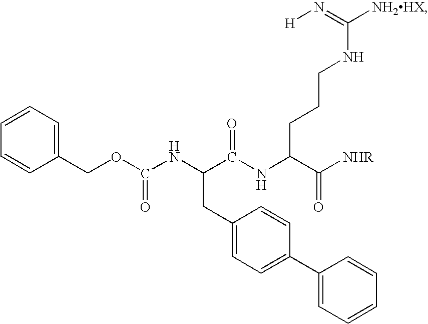
wherein HX is a mineral acid salt or organic acid salt; and wherein R is preferably H or an unsubstituted or substituted alkyl or an unsubstituted or substituted aryl.

Embodiments provide a compound having the formula: 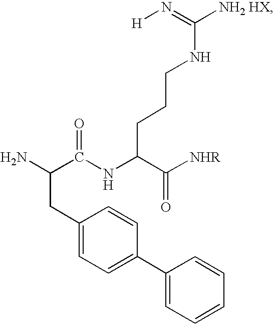
wherein HX is a mineral acid salt or organic acid salt; and wherein R is preferably H or an unsubstituted or substituted alkyl or an unsubstituted or substituted aryl.

Embodiments provide a compound having the formula: 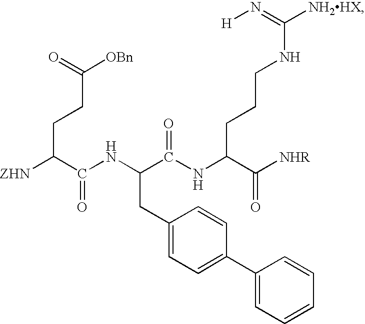
wherein HX is a mineral acid salt or organic acid salt; and wherein R is preferably H or an unsubstituted or substituted alkyl or an unsubstituted or substituted aryl.

Embodiments provide a compound having the formula: 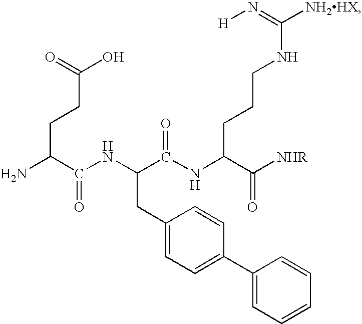
wherein HX is a mineral acid salt or organic acid salt; and wherein R is preferably H or an unsubstituted or substituted alkyl or an unsubstituted or substituted aryl.

Embodiments provide a compound having the formula: 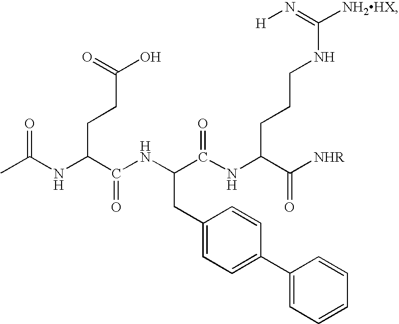
wherein HX is a mineral acid salt, organic acid salt or pharmaceutically acceptable salt; and wherein R is preferably H or an unsubstituted or substituted alkyl or an unsubstituted or substituted aryl.

### DETAILED DESCRIPTION OF THE PREFERRED EMBODIMENT

The mediators of RCT obtained by embodiments are molecules comprising three regions, an acidic region, a hydrophobic or lipophilic (e.g., aromatic) region, and a basic region. The molecules preferably contain a positively charged region, a negatively charged region, and an uncharged, lipophilic region. The locations of the regions with respect to one another can vary between molecules; thus, in an embodiment, the molecules mediate RCT regardless of the relative positions of the three regions within each molecule. As used herein, the terms “mediator” and “facilitator” are interchangeable.

Embodiments provide molecular mediators of RCT comprising trimers of natural D- or L-amino acids, amino acid analogs (synthetic or semisynthetic), and amino acid derivatives. For example, a trimer includes an acidic amino acid residue or analog thereof, an aromatic or lipophilic amino acid residue or analog thereof, and a basic amino acid residue or analog thereof, the residues being joined by peptide or amide bond linkages. For example, the trimer sequence EFR comprises an acidic residue (glutamic acid), an aromatic residue (phenylalanine) and a basic amino acid residue (arginine). The acidic-aromatic-basic trimer sequence can comprise EFR or efr or rfe, i.e., containing D-amino acid residues or E-(4-Phenyl)-FR or modified or synthetic or semisynthetic amino acid residues.

While the molecular mediators of RCT share the common aspect of reducing serum cholesterol, probably through enhancing direct and/or indirect RCT pathways (i.e., increasing cholesterol efflux), the mediators may exhibit inter alia one or more of the following specific functional attributes: ability to form amphipathic helical structures or sub-structures thereof in the presence or absence of lipid, ability to bind lipids, ability to form pre-β-like or HDL-like complexes, ability to activate LCAT, and ability to increase serum HDL concentration.

Thus, embodiments include methods for synthesizing short and stable peptide mediators of RCT that preferably exhibit preferential lipid binding conformation, increase cholesterol flux to the liver by facilitating direct and/or indirect reverse cholesterol transport, improve the plasma lipoprotein profile, and subsequently prevent the progression or/and even promote the regression of atherosclerotic lesions.

As used herein, the term “peptide” can include peptides, peptide analogs, and peptide derivatives. An analog is a structural derivative of a parent compound that may differ from the parent by one or more elements. A derivative is a compound derived or obtained from another and containing integral elements of the parent substance.

As used herein, the abbreviations for the genetically encoded L-enantiomeric amino acids are conventional and are as follows: The D-amino acids are designated by lower case, e.g. D-alanine=a, etc.

TABLE 1

| Amino Acids | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Certain amino acid residues in the peptide mediators of RCT can be replaced with other amino acid residues without significantly deleteriously affecting, and in many cases even enhancing, the activity of the peptides. Thus, also contemplated by the embodiments are altered or mutated forms of the peptide mediators of RCT wherein at least one defined amino acid residue in the structure is substituted with another amino acid residue or derivative and/or analog thereof. As one of the features affecting the activity of the peptides of the embodiments may be their ability to form a-helices in the presence of lipids that exhibit the amphipathic and other properties described above, it will be recognized that in embodiments, the amino acid substitutions are conservative, i.e., the replacing amino acid residue has physical and chemical properties that are similar to the amino acid residue being replaced.

For purposes of determining conservative amino acid substitutions, the amino acids can be conveniently classified into two main categories—hydrophilic and hydrophobic—depending primarily on the physical-chemical characteristics of the amino acid side chain. These two main categories can be further classified into subcategories that more distinctly define the characteristics of the amino acid side chains. For example, the class of hydrophilic amino acids can be further subdivided into acidic, basic and polar amino acids. The class of hydrophobic amino acids can be further subdivided into nonpolar and aromatic amino acids. The definitions of the various categories of amino acids that define ApoA-I are as follows:

The term “hydrophilic amino acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gln (Q), Asp (D), Lys (K) and Arg (R).

The term “hydrophobic amino acid” or “lipophilic amino acid” refers to an amino acid exhibiting a hydrophobicity of greater than zero according to the normalized consensus hydrophobicity scale of Eisenberg, 1984, J. Mol. Biol. 179:1.25-142. Genetically encoded hydrophobic amino acids include Pro (P), Ile (I), Phe (F), Val (V), Leu (L), Trp (W), Met (M), Ala (A), Gly (G) and Tyr (Y).

The term “acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

The term “basic amino acid” refers to a hydrophilic amino acid having a side chain pK value of greater than 7. Basic amino acids typically have positively charged side chains at physiological pH due to association with hydronium ion. Genetically encoded basic amino acids include His (H), Arg (R) and Lys (K).

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gin (Q) Ser (S) and Thr (T).

The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aromatic amino acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —(O)NH₂, —C(O)NHR, —C(O)NRR and the like where each R is independently (C₁-C₆)alkyl, substituted (C₁-C₆)alkyl, (C₁-C₆)alkenyl, substituted (C₁-C₆)alkenyl, (C₁-C₆) alkynyl, substituted (C₁₆)alkynyl, (C₅-C₂₀)aryl, substituted (C₅-C₂₀)aryl, (C₆-C₂₆)alkaryl, substituted (C₆-C₂₆)alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe (F), Tyr (Y) and Trp (W).

The term “aliphatic amino acid” refers to a hydrophobic amino acid having an aliphatic hydrocarbon side chain. Genetically encoded aliphatic amino acids include Ala (A), Val (V), Leu (L) and Ile (I).

Certain commonly encountered amino acids which provide useful substitutions for the peptide mediators of RCT include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Mehle); phenylglycine (Phg); cyclohexylalanine (Cha); norleucine (Nle); naphthylalanine (Nal); 4-phenylphenylalanine, 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe (pNH₂)); N-methyl valine (MeVal); homocysteine (hCys), homophenylalanine (hphe) and homoserine (hSer); hydroxyproline (Hyp), homoproline (hPro), N-methylated amino acids and peptoids (N-substituted glycines).

Other amino acid residues not specifically mentioned herein can be readily categorized based on their observed physical and chemical properties in light of the definitions provided herein.

The classifications of the genetically encoded and common non-encoded amino acids according to the categories defined above are summarized in Table 2, below. It is to be understood that Table 2 is for illustrative purposes only and does not purport to be an exhaustive list of amino acid residues and derivatives that can be used to substitute the peptide mediators of RCT described herein.

TABLE 2
CLASSIFICATIONS OF COMMONLY
ENCOUNTERED AMINO ACIDS

| | | |
|---|---|---|
| Classification | Genetically Encoded | Non-Genetically Encoded |

Hydrophobic

| | | |
|---|---|---|
| Aromatic | F, Y, W | Phg, Nal, Thi, Tic, Phe (4-Cl), |
| | | Phe (2-F), Phe (3-F), Phe (4-F), |
| | | hPhe |
| Nonpolar | L, V, I, M, G, A, P | t-BuA, t-BuG, MeIle, Nle, |
| | | MeVal, Cha, McGly, Aib |
| Aliphatic | A, V, L, I | b-Ala, Dpr, Aib, Aha, MeGly, |
| | | t-BuA, t-BuG, MeIle, Cha, Nle, |
| | | MeVal |

Hydrophilic

| | | |
|---|---|---|
| Acidic | D, E | |
| Basic | H, K, R | Dpr, Orn, hArg, Phe(p-NH₂), |
| | | Dbu, Dab |
| Polar | C, Q, N, S, T | Cit, AcLys, MSO, bAla, hSer |
| Helix-Breaking | P, G | D-Pro and other D-amino acids |
| | | (in L-peptides) |

Other amino acid residues not specifically mentioned herein can be readily categorized based on their observed physical and chemical properties in light of the definitions provided herein.

While in many instances, the amino acids of the peptide mediators of RCT will be substituted with L-enantiomeric amino acids, the substitutions are not limited to L-enantiomeric amino acids. Thus, also included in the definition of “mutated” or “altered” forms are those situations where an L-amino acid is replaced with an identical D-amino acid (e.g., L-Arg-D-Arg) or with a D-amino acid of the same category or subcategory (e.g., L-Arg-D-Lys), and vice versa. Indeed, in certain embodiments that are suitable for oral administration to animal subjects, the peptides may advantageously be composed of at least one D-enantiomeric amino acid. Peptides containing such D-amino acids are thought to be more stable to degradation in the oral cavity, gut or serum than are peptides composed exclusively of L-amino acids. Compounds herein that do not indicate stereochemistry at a chiral center of an amino acid encompass both possibilities of D and L amino acids. In one embodiment, the compounds described herein may be present as mixtures of distereoisomers or enantiomers.

The mediators of RCT can be further defined by way of embodiments. In one embodiment, there is a molecule comprising an amino acid-based composition having three independent regions: an acidic region, an aromatic or lipophilic region, and a basic region. Thus, a trimeric peptide in accordance with this embodiment, such as EFR, or erf or fre contains an acidic amino acid residue, an aromatic or lipophilic residue and a basic residue. The relative locations of the regions with respect to one another can vary between molecular mediators; the molecules mediate RCT regardless of the position of the three regions within each molecule. In mediators comprising a trimeric peptide, such as EFR or efr, the trimers may consist of natural D- or L-amino acids, amino acid analogs, and amino acid derivatives.

In another embodiment, the aromatic region of the trimer may consist of nicotinic acid with an acidic or basic side chain(s).

In another embodiment, the aromatic region of the trimer may consist of 4-phenyl phenylalanine.

As used herein, a protecting group is a group that is used to protect a functional group from undesired reactions. After application, the protecting group can be removed to reveal the original functional group. A capping group is also a group that is used to protect a functional group but that is preferably retained in the compound. Certain groups, e.g., acetyl, may act as both a protecting group and a capping group.

In another variation, an N-terminal protecting group PG¹ comprises a group R^{x}—CO— wherein R^{x} is selected from the group consisting of methyl, phenyl-CH₂—, di-tert-butyl-4-hydroxy-phenyl, naphthyl, substituted naphthyl, 9-fluorenylmethoxy-, biphenyl, substituted phenyl, substituted heterocycles, alkyl, aryl, substituted aryl, cycloalkyl, fused cycloalkyl, saturated heteroaryl, substituted saturated heteroaryl and the like. A particular value for PG¹ is acetyl.

In another variation, the C-terminal protecting group PG is an amino (NH₂) group or a group —NHR^{y} where R^{y}=di-tert-butyl-4-hydroxy-phenyl, naphthyl, substituted naphthyl, Fmoc, biphenyl, substituted phenyl, substituted heterocycles, alkyl, aryl, substituted aryl, cycloalkyl, fused cycloalkyl, saturated heteroaryl, substituted saturated heteroaryl, and the like. A particular value for PG² is NH₂.

In another embodiment, the protecting group PG³ is benzyloxy carbonyl.

In another embodiment, the protecting group PG⁴ is benzyloxy carbonyl.

In another embodiment, PG² is benzyloxycarbonyl, PG⁴ is benzyloxy carbonyl and R³ is benzyloxycarbonylethyl.

A schematic representation of an embodiment is shown below in Synthetic Scheme 1. Variations in protecting groups, activating reagents, and coupling reagents are expected.

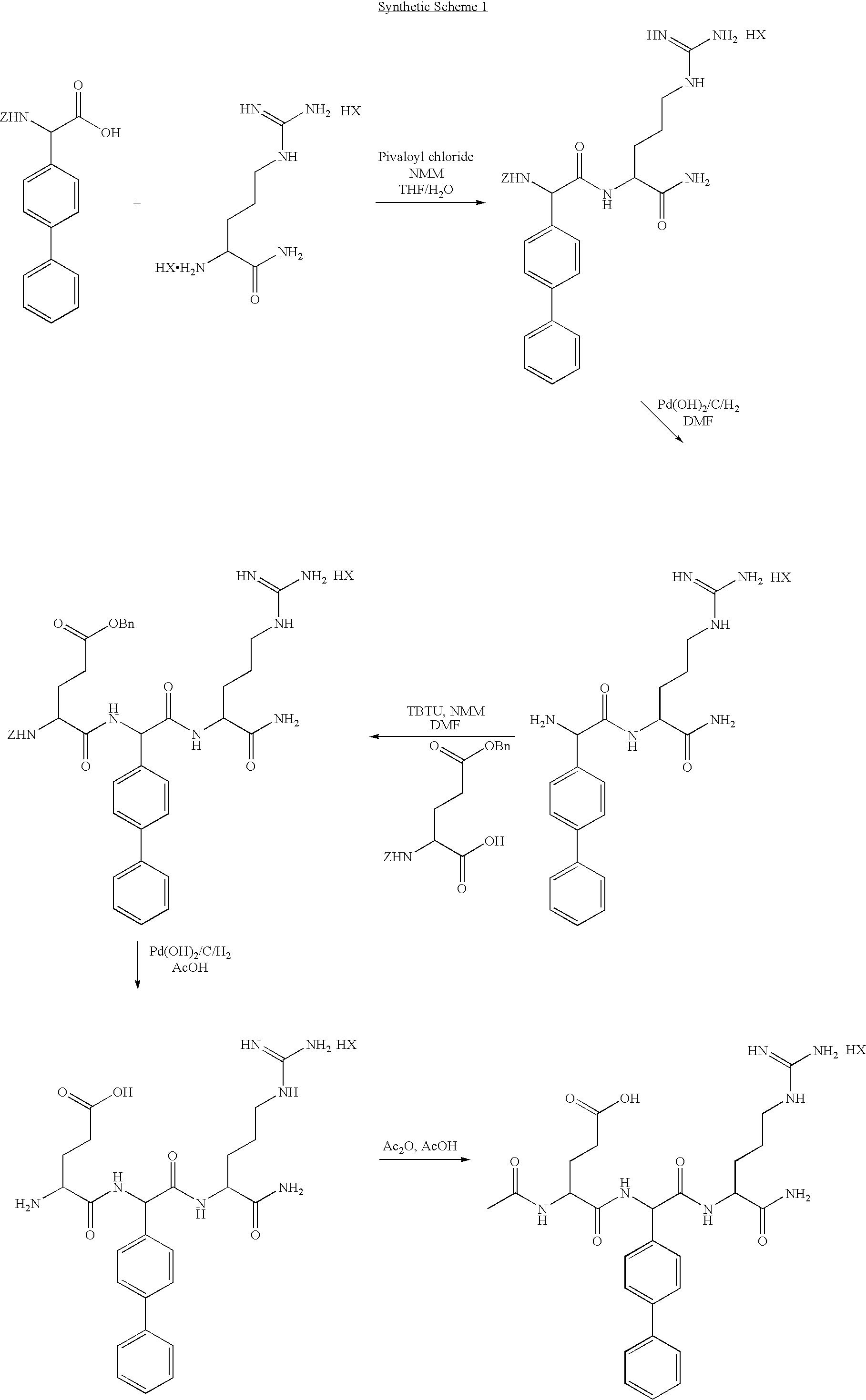

Preferably, the N terminus is acetylated using Ac₂O. However, in other preferred embodiments, the N-terminus is acylated with RCO]₂O or RCOCl, where R is an acyl group of 2-30 carbons, preferably, 2-10 carbons, more preferably 2-5 carbons.

HX represents a salt. Some examples of pharmaceutically acceptable salts that can be used in the embodiments include those salt-forming acids and bases which do not substantially increase the toxicity of the compound. Salts of mineral acids include hydrochloric, hydriodic, hydrobromic, phosphoric, metaphosphoric, nitric and sulfuric acids, as well as salts of organic acids such as. tartaric, acetic, citric, malic, benzoic, succinic, arylsulfonic, e.g. p-toluenesulfonic acids, and the like. A particularly suitable value for HX is HCl.

The stereochemistry of the compounds in the Synthetic Scheme 1 can include D or L amino acids. An embodiment is Ac-e-bip-r-NH₂ in which all the amino acids are D.

Synthetic Scheme 2 shows a generic version of the synthetic scheme for a tripeptide. In Synthetic Scheme 2, protecting groups are represented by PG. Amino acid side chains are represented by R. In embodiments, the substituents for R¹, R², R³, PG¹, PG², PG³, and PG⁴ are disclosed in Table 3.

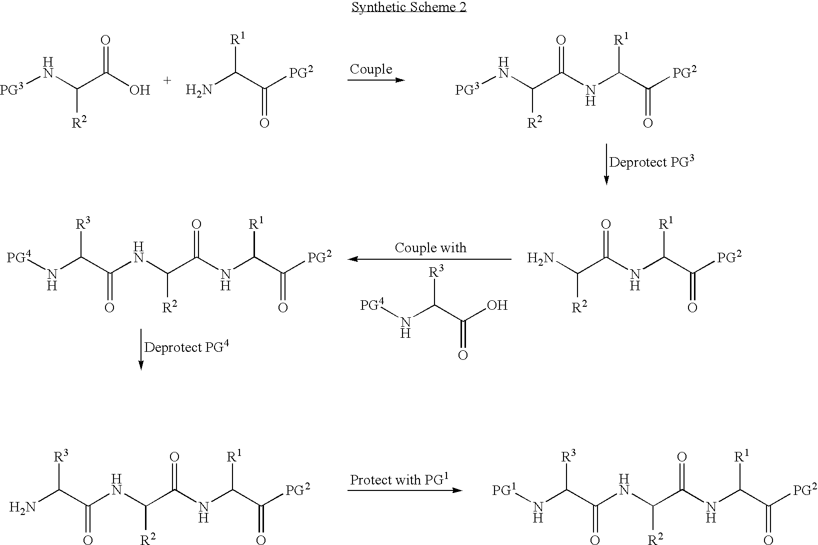

In Synthetic Scheme 2, an amino acid is protected at the carboxyl end and another amino acid is protected at the amino end. Both of these amino acids can be coupled to each other. In some instances, the coupling reaction can occur with the aid of a coupling agent. The product of this coupling reaction is a dipeptide in which both the amino end and carboxyl end are protected. Further addition of amino acids to the peptide can occur with deprotection of one end of the peptide and coupling an amino acid to the peptide.

A coupling reagent can be any reagent known in the peptide synthesis field to aid in coupling peptides and/or amino acids. Some common peptide coupling reagents which may be used are, for example, acid chlorides (and acyl halides), acyl azides, acyl imidazoles, anhydrides, carbodiimide reagents (to which additives such as HOBt or NCS may be added), the HOBt family of reagents, active esters, phosphonium reagents, uranium reagents, ammonium salts and certain enzymes. Such reagents and the reaction conditions used for coupling reactions are well known in the art of peptide synthesis, and are disclosed in standard texts concerned with peptide synthesis such as Bodansky and Bodansky, The Practice of Peptide Synthesis, Springer, 2^{nd} Edition, 1994, or in published papers such as Montalbetti and Falque, Tetrahedron 2005, 61, 10827-10852, incorporated herein by reference.

Care has to be taken in selection of protecting groups so that they can be removed with appropriate selectivity. Side chain and carboxyl protecting groups need to be selected so as not to be labile under the conditions required to deprotect the amino group.

Two ways of achieving selective deprotection are 1) choosing protecting groups that are deprotected with completely different reagents (referred to as orthogonal protection) e.g. tert butyl (acid), fluorenyl methyl (base), benzyl (catalytic hydrogenolysis) and 2) choosing protecting groups that are deprotected with the same type of reagent but under different conditions. e.g. tert butyl and benzyl which require increasingly strong acids for deprotection.

In embodiments, protecting groups can be left on the molecule. Alternatively, protecting groups can be exchanged for each other. Standard procedures are available in the art for exchanging protecting groups. For example, protecting groups are discussed in T. W. Greene and P. G. M. Wuts, Protective Groups in Organic Synthesis, 3rd Edition, (1999) John Wiley & Sons.

An amino end protecting group can be any reagent known in the art to protect the amino group functionality, preferably in the peptide synthesis field. Some common amino end protecting groups are alkoxycarbonyl or substituted alkoxycarbonyl protecting groups, such as benzyloxycarbonyl (Z or Cbz), t-butoxycarbonyl (Boc), 2-(4-biphenyl)-isopropoxycarbonyl (Bpoc) and 9-fluorenylmethoxycarbonyl (Fmoc). Other include silicon reagents, such as triphenylmethyl (trityl), and 2-nitrophenylsulfenyl (Nps). In the embodiment shown in Synthetic Scheme 1, benzyloxycarbonyl is particularly suitable. In embodiments, the amino end protecting groups are recited in Table 3.

In the above scheme, an amide group can act as a protecting group for the carboxyl end. A carboxyl end protecting group can be any reagent known in the art to protect the carboxyl group functionality, preferably in the peptide synthesis field. Some common carboxyl end protecting groups are esters, for example (1-4C alkyl esters such as methyl, ethyl and t-butyl esters. Other esters include for example, substituted or unsubstituted phenyl or benzyl esters. In embodiments, the carboxyl end protecting groups are recited in Table 3.

In Synthetic Scheme 1, the combination of protecting groups and/or coupling agents provide for optimization of the synthetic scheme. In this synthetic scheme, benzyloxycarbonyl and benzyl groups are used as protecting groups. These groups can be deprotected with hydrogenation. Previously experimented protecting groups of t-Boc and t-butyl ester did not provide results matching those of benzyloxycarbonyl and benzyl groups.

In this synthetic scheme, pivaloyl chloride and O-(Benzotriazol-1-yl)-N,N,N′,N′-tetramethyluronium tetrafluoroborate (TBTU) are used as coupling reagents. The combination of pivaloyl chloride and TBTU also provides optimized conditions for the coupling reactions. In the coupling steps, other bases besides N-methylmorpholine (NMM) may be used, including for example, diisopropylethylamine and triethylamine.

In Synthetic Scheme 1, it is particularly useful to be able to use the argininamide in the coupling reaction as its salt, without the need to have a protecting group covalently bonded to the guanidine group, which would need to be removed at a later stage. It is also particularly useful to use argininamide as the carboxylic end of the molecule is already protected with the desired NH₂ group. It is also particularly useful to use THF as solvent in this coupling step.

The embodiments provide for a method of preparing the compounds using solution phase peptide synthesis.

Methods for acylating the N-terminus of a peptide or amidating or esterifying the C-terminus of a peptide are well-known in the art. Modes of carrying other modifications at the N- and/or C-terminus will be apparent to those of skill in the art, as will modes of protecting any side-chain functionalities as may be necessary to attach terminal blocking groups.

Pharmaceutically acceptable salts (counter ions) can be conveniently prepared by ion-exchange chromatography or other methods as are well known in the art.

Peptides which may be prepared by the processes of the embodiments are shown in the following Table 3.

TABLE 3

| Compound | | MOL. | MOL. |
|---|---|---|---|
| # | SEQUENCE | FORMULA | WEIGHT |
| 3-1 | Ac-E-F—R—NH₂ | C₂₂H₃₃N₇O₆ | 491.5 |
| 3-2 | Ac-e-r-f-NH₂ | C₂₂H₃₃N₇O₆ | 491.5 |
| 3-3 | Ac-E-BIP—R—NH₂ | C₂₈H₃₇N₇O₆ | 567.6 |
| 3-4 | 2-Naph-E-F—R—NH₂ | C₃₁H₃₇N₇O₆ | 603.6 |
| 3-5 | 2-Nap-e-r-f-NH₂ | C₃₁H₃₇N₇O₆ | 603.6 |
| 3-6 | Piv-E-F—R—NH₂ | C₂₅H₃₉N₇O₆ | 533.6 |
| 3-7 | NA-E-F—R—NH₂ | C₂₆H₃₄N₈O₆ | 554.6 |
| 3-8 | Ac-r-f-e-NH₂ | C₂₂H₃₃N₇O₆ | 491.5 |
| 3-9 | Fmoc-E-F—R—OH | C₃₅H₄₀N₆O₈ | 672.7 |
| 3-10 | NA-E-BIP—R—NH₂ | C₃₂H₃₈N₈O₆ | 630.7 |
| 3-11 | 2-Nap-E-BIP—R—NH₂ | C₃₇H₄₁N₇O₆ | 679.7 |
| 3-12 | NA-e-f-r-NH₂ | C₂₆H₃₄N₈O₆ | 554.6 |
| 3-13 | 3,5-di-t-butyl-4-OH-PhCO-E-BIP—R—NH₂ | C₄₁H₅₅N₇O₇ | 757.9 |
| 3-14 | NA-f-r-e-NH₂ | C₂₆H₃₄N₈O₆ | 554.6 |
| 3-15 | 1-Nap-E-BIP—R—NH₂ | C₃₇H₄₁N₇O₆ | 679.7 |
| 3-16 | NA-e-r-f-NH₂ | C₂₆H₃₄N₈O₆ | 554.6 |
| 3-17 | Ac-e-BIP-r-NH₂ | C₂₈H₃₇N₇O₆ | 567.6 |
| 3-18 | Ac-e-bip-r-NH₂ | C₂₈H₃₇N₇O₆ | 567.6 |
| 3-19 | Isoxazole-e-bip-r-NH₂ | C₃₁H₃₈N₈O₇ | 634.6 |
| 3-20 | Ac-E-BIP—R—OH | C₃₂H₃₈N₈O₆ | 568.6 |
| 3-21 | 3,5-di-t-butyl-4-OH-PhCO-e-r-f-NH₂ | C₃₅H₅₁N₇O₇ | 681.8 |
| 3-22 | 3,5-di-t-butyl-4-OH-PhCO-f-e-r-NH₂ | C₃₅H₅₁N₇O₇ | 681.8 |
| 3-23 | Isoxazole-f-e-r-NH₂ | C₃₅H₅₁N₇O₇ | 558.5 |
| 3-24 | NA-f-e-r-NH₂ | C₂₆H₃₄N₈O₆ | 554.6 |
| 3-25 | Isoxazole-r-f-e-NH₂ | C₃₅H₅₁N₇O₇ | 558.5 |
| 3-26 | 1-Nap-r-f-e-NH₂ | C₃₁H₃₇N₇O₆ | 603.6 |
| 3-27 | NA-r-f-e-NH₂ | C₂₆H₃₄N₈O₆ | 554.6 |
| 3-28 | Ac-E-F(4-I)—R—NH₂ | C₂₂H₃₂IN₇O₆ | 617.4 |
| 3-29 | NA-R—BIP-E-NH₂ | C₃₂H₃₈N₈O₆ | 630.7 |
| 3-30 | 1-Nap-R—BIP-E-NH₂ | C₃₇H₄₁N₇O₆ | 679.7 |
| 3-31 | Ac—R—BIP-E-NH₂ | C₂₈H₃₇N₇O₆ | 567.6 |
| 3-32 | Ac-E-BIP-K—NH₂ | C₂₈H₃₇N₅O₆ | 539.6 |
| 3-33 | Ac-D-BIP-K—NH₂ | C₂₇H₃₅N₅O₆ | 525.6 |
| 3-34 | Ac-d-BIP-r-NH₂ | C₂₇H₃₇N₅O₆ | 553.6 |
| 3-35 | Ac-e-bip-k-NH₂ | C₂₈H₃₇N₅O₆ | 539.6 |
| 3-36 | Ac-d-bip-k-NH₂ | C₂₇H₃₅N₅O₆ | 525.6 |
| 3-37 | Ac-d-bip-r-NH₂ | C₂₇H₃₇N₅O₆ | 553.6 |
| 3-38 | Ac-E-R—BIP—NH₂ | C₂₈H₃₇N₇O₆ | 567.6 |
| 3-39 | 2-Pyrazine-E-R—BIP—NH₂ | C₃₁H₃₇N₉O6 | 631.6 |
| 3-40 | Piperonylic-E-R—BIP—NH₂ | C₃₄H₃₉N₇O₈ | 673.7 |
| 3-41 | Ac-E-bip-R—NH₂ | C₂₈H₃₇N₇O₆ | 567.6 |
| 3-42 | Ac-e-f(4-I)-r-NH₂ | C₂₂H₃₂IN₇O₆ | 617.4 |
| 3-43 | Ac-e-bip(4-tBu)-r-NH₂ | C₃₂H₄₅N₇O6 | 673.7 |
| 3-44 | Ac-E-BIP(4-tBu)-R—NH₂ | C₃₂H₄₅N₇O₆ | 673.7 |
| 3-45 | Ac-e-bip(4-CF₃)-r-NH₂ | C₂₉H₃₆F₃N₇O₆ | 635.6 |
| 3-46 | Ac-e-bip(2,6-dichloro)-r-NH₂ | C₂₈H₃₅Cl₂N₇O₆ | 636.5 |
| 3-47 | Ac-E-BIP(2,6-dichloro)-R—NH₂ | C₂₈H₃₅Cl₂N₇O₆ | 636.5 |
| 3-48 | Ac-e-Aic-r-NH₂ | C₂₃H₃₃N₇O₆ | 503.5 |
| 3-49 | Ac-E-Aic-R—NH2 | C₂₃H₃₃N₇O₆ | 503.5 |
| 3-50 | Ac-e-3pa-r-NH₂ | C₂₁H₃₂N₈O₆ | 492.5 |
| 3-51 | Ac-E-3PA-R—NH₂ | C₂₁H₃₂N₈O₆ | 492.5 |
| 3-52 | Ac-E-bip-r-NH₂ | C₂₈H₃₇N₇O₆ | 567.6 |
| 3-53 | Ac-e-bip-R—NH₂ | C₂₈H₃₇N₇O₆ | 567.6 |
| 3-54 | Ac-E-BIP[4-(2-Nap)]-R—NH₂ | C₃₂H₃₉N₇O₆ | 617.6 |
| 3-55 | Ac-e-f((4-)3-Py))-r-NH₂ | C₂₇H₃₆N₈O₆ | 568.6 |
| 3-56 | Ac-E-4PA-R—NH₂ | C₂₁H₃₂N₈O₆ | 492.5 |
| 3-57 | Ac-E-2PA-R—NH₂ | C₂₁H₃₂N₈O₆ | 492.5 |
| 3-58 | Ac-E-F((4-)3-Py))-R—NH₂ | C₂₇H₃₆N₈O₆ | 568.6 |
| 3-59 | Ac-d-Aic-r-NH₂ | C₂₂H₃₁N₇O₆ | 489.5 |
| 3-60 | Ac-E-W-R—NH₂ | C₂₄H₃₄N₈O₆ | 530.5 |
| 3-61 | Ac-e-w-r-NH₂ | C₂₄H₃₄N₈O₆ | 530.5 |
| 3-62 | Ac-E-F(4-BIP)—R—NH₂ | C₃₄H₄₇N₇O₆ | 643.7 |
| 3-63 | Ac-E(O-chx)-F—R—NH₂ | C₂₈H₄₃N₇O₆ | 573.7 |
| 3-64 | 3,5-di-t-butyl-4-OH-PhCO--r-f-e-NH₂ | C₃₅H₅₁N₇O₇ | 681.8 |
| 3-65 | Ac-E-F—R(NO₂)—NH₂ | C₂₂H₃₂N₈O₈ | 536.5 |
| 3-66 | Ac-e(O-t-butyl)-bip-r-NH₂ | C₃₂H₄₅N₇O₆ | 623.7 |
| 3-67 | Biotinyl-e-bip-r-NH₂ | C₃₆H₄₉N₉O₇S | 751.7 |
| 3-68 | 3,5-di-t-butyl-4-OH-PhCO-r-f-e-NH₂ | C₄₁H₅₅N₇O₇ | 757.7 |
| 3-69 | Biotinyl-e-f-r-NH₂ | C₃₀H₄₅N₉O₇S | 675.8 |
| 3-70 | Ac-E-BIP—R(—NO₂)—NH₂ | C₂₈H₃₆N₈O₈ | 612.6 |
| 3-71 | Ac-E(tetrazole)-F—R—NH₂ | C₂₃H₃₄N₁₂O₅ | 558.6 |
| 3-72 | Ac-e(tetrazole)-f-r-NH₂ | C₂₃H₃₄N₁₂O₅ | 558.6 |
| 3-73 | Ac-E(tetrazole)-BIP—R—NH₂ | C₂₉H₃₈N₁₂O₅ | 634.7 |
| 3-74 | Ac-e(tetrazole)-bip-r-NH₂ | C₂₉H₃₈N₁₂O₅ | 634.7 |
| 3-75 | Ac-E-F—R(—CN)—NH₂ | C₂₃H₃₂N₈O₆ | 516.5 |
| 3-76 | Ac-e-f-r(-CN)—NH₂ | C₂₃H₃₂N₈O₆ | 516.5 |
| 3-77 | Ac-E-BIP—R(—CN)—NH₂ | C₂₉H₃₆N₈O₆ | 592.6 |
| 3-78 | Ac-e-bip-r(—CN)—NH₂ | C₂₉H₃₆N₈O₆ | 592.6 |
| 3-79 | Ac-E(tetrazole)-F—R(NO₂)—NH₂ | C₂₃H₃₃N₁₃O₇ | 603.6 |
| 3-80 | Ac-e(tetrazole)-f-r(NO₂)—NH₂ | C₂₃H₃₃N₁₃O₇ | 603.6 |
| 3-81 | Ac-E(tetrazole)-BIP—R(NO₂)—NH₂ | C₂₉H₃₇N₁₃O₇ | 679.7 |
| 3-82 | Ac-e(tetrazole)-bip-r(NO₂)—NH₂ | C₂₉H₃₇N₁₃O₇ | 679.7 |
| 3-83 | Ac-E(tetrazole)-F—R(CN)—NH₂ | C₂₄H₃₃N₁₃O₅ | 583.6 |
| 3-84 | Ac-e(tetrazole)-f-r(CN)—NH₂ | C₂₄H₃₃N₁₃O₅ | 583.6 |
| 3-85 | Ac-E(tetrazole)-BIP—R(CN)—NH₂ | C₃₀H₃₇N₁₃O₅ | 659.7 |
| 3-86 | Ac-e(tetrazole)-bip-r(CN)—NH₂ | C₃₀H₃₇N₁₃O₅ | 659.7 |
| 3-87 | Ac-e-f-r(-NO₂)—NH₂ | C₂₂H₃₂N₈O₈ | 536.5 |
| 3-88 | Ac-e-bip-r(-NO₂)—NH₂ | C₂₈H₃₆N₈O₈ | 612.6 |
| 3-89 | 3,5-di-t-butyl-4-OH-PhCO-e-f-r(-CN)—NH₂ | C₃₆H₅₀N₈O₇ | 706.8 |
| 3-90 | 3,5-di-t-butyl-4-OH-PhCO-e(tetrazole)-f- | C₃₇H₅₁N₁₃O₆ | 773.8 |
| | r(-CN)—NH₂ | | |
| 3-91 | Ac-E(tetrazole)-F-K—NH₂ | C₃₅H₅₁N₅O₇ | 653.8 |
| 3-92 | 3,5-di-t-butyl-4-OH-PhCO-E(tetrazole)-F— | C₃₆H₅₁N₁₃O₈ | 793.8 |
| | R(—NO₂)—NH₂ | | |
| 3-93 | Ac-e-f-c-NH₂ | C₁₉H₁₆N₄O₆S | 438.5 |
| 3-94 | Ac-e-bip-c-NH₂ | C₂₅H₃₀N₄O₆S | 514.6 |
| 3-95 | (Ac-e-bip-c-NH₂)-Dimer | C₅₀H₅₈N₈O₁₂S₂ | 1027.1 |
| 3-96 | (Ac-e-f-c-NH₂)-Dimer | C₃₈H₃₀N₈O₁₂S₂ | 875 |
| 3-97 | Ac-EYR—NH₂ | | |
| 3-98 | Ac-E-(L-2-p-Naphthylalanine)-R—NH₂ | | |
| 3-99 | Ac-E-(L-1-3-Naphthylalanine)-R—NH₂ | | |
| 3-100 | H-EFR—OH | | |
| 3-101 | Ac-EFR—OH | | |
| 3-102 | H-EFR—NH₂ | | |
| 3-103 | Ac—RFE-NH₂ | | |
| 3-104 | Ac-efr-NH₂ | | |

The abbreviations used for the D-enantiomers of the genetically encoded amino acids are lower-case equivalents of the one-letter symbols shown in Table 1. For example, “R” designates L-arginine and “r” designates D-arginine.

Tetrazole denotes tetrazole-amine-amide.

Unless otherwise defined, the N-terminus (left side) is capped with an RCO group, where RCO or R is defined as follows:

Ac denotes acetylated.

Piv denotes pivolylated.

1-Nap & 2-Nap indicate naphthylic acid capped.

Fmoc denotes an N-terminus modified with 9-fluorenylmethyloxycarbonyl.

NA denotes nicotinic acid.

BIP denotes biphenylalanine. In most instances, it is understood that BIP denotes 4-biphenylalanine.

Isoxazole or Isox denotes 5-methyl-isoxazole-3-carboxylic acid derivative.

Pyrazine denotes the carboxylic acid derivative.

Aic denotes 2-amino, 2-carboxy indane.

PA denotes (2, 3 or 4)-pyridyl alanine.

Py denotes pyridine.

O—Chx denotes a cyclohexyl ester.

### EXAMPLES

The disclosure below is of specific examples setting forth methods. These examples are not intended to limit the scope, but rather to exemplify embodiments.

### Example 1

### Synthesis of Certain Compounds

### Stage 1: Coupling of Z-(D)BIP with H-(D)Arg-NH₂.2HCl

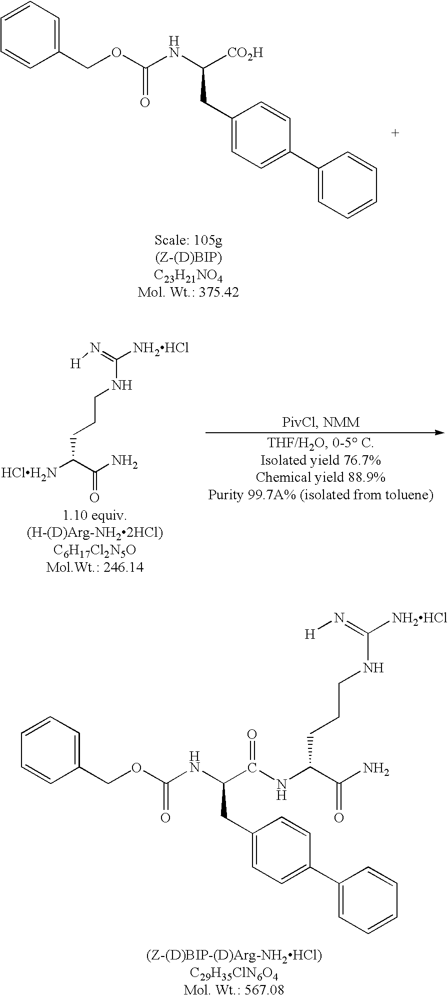

The mixed anhydride Z-(D)BIP-OPiv was prepared by slowly (over 40 minutes) adding N-methylmorpholine (NMM) (Sigma-Aldrich 99%, 29.4 g, 1.04 eq.) to a chilled (<5° C.) mixture of Z-(D)BIP (Synthetec, 105 g) and pivaloyl chloride (PivCl) (Aldrich 99%, 35.4 g, 1.05 eq.) in THF (Sigma-Aldrich 99+%, 630 mL, 6 volumes vs Z-(D)BIP). The reaction was somewhat exothemic but easily addition controlled. The reaction mixture was stirred for an additional 30 minutes at <5° C.

The cold (<5° C.) solution of mixed anhydride Z-(D)BIP-OPiv was slowly (over 26 minutes) added to a cold solution of H-(D)Arg-NH₂.2HCl (Bachem, 75.7 g, 1.1 eq.) in deionized water (303 mL) and NMM (Sigma-Aldrich 99%, 31.1 g, 1.10 eq.). The reaction mixture temperature was maintained below 5° C. The reaction was exothermic but easily addition controlled. The reaction was stirred at <5° C. overnight.

The liquid-liquid biphasic reaction mixture was allowed to warm-up to room temperature and the organic phase was separated.

The aqueous layer was eliminated and the cloudy organic phase was extracted with a mixture of 10% aqueous NaCl solution (263 niL, NaCl quality USP) and NMM (24 mL). The lower layer volume was 375 mL (pH z 7-7.5, pH paper). The upper layer volume was 790 mL.

The aqueous layer was eliminated and the organic phase was extracted with a mixture of 10% aqueous NaCl solution (263 mL) and aqueous HCl 18% (32 mL).

After the aqueous layer was eliminated, the reactor jacket temperature was adjusted to 42.5° C. (to prevent solidification of the organic layer). A portion of the previously obtained organic layer was reintroduced into the 3 L jacketed vessel (132 g out of 661 g) and allowed to warm to 42° C. A mixture of 10% aqueous NaCl solution (19 mL) and THF (149 mL) was added. Then, toluene (Sigma-Aldrich 99%, 280 mL) was added slowly (over 5 minutes) to the mixture while maintaining the reaction mixture temperature above 37° C. The mixture was well stirred for 10 minutes and then the layers were allowed to settle. A liquid-liquid-liquid triphasic mixture was obtained. The lower layer was removed.

More THF (70 mL) was added to the liquid-liquid biphasic system. Upon stirring, a clear, single liquid layer appeared. This mixture of THF, water and toluene was next distilled under vacuum. The reaction mixture was reduced from ≈530 mL to ≈420 mL under 190-150 τ at 37-39° C. in the vessel (jacket temperature: 42.5-47.5° C.). During this operation, the reaction mixture turned into a slurry.

Upon overnight cooling under agitation, a slurry was formed. The slurry was filtered over filter paper (Whatman No 3, 110 mm diameter). The wet cake was then reslurried twice in methyl tert-butyl ether (MTBE) (high purity, 75 mL each time) on the Buchner filter. After 3 days drying in the oven (150 r, room temperature, nitrogen flow), 27.0 g of Z-(D)BIP-(D)Arg-NH₂.HCl was obtained.

The remaining portion of the organic layer (530 g) was treated under similar conditions at ≈40° C.: A mixture of 10% aqueous NaCl solution (88 mL) and THF (588 mL) was added. Then, toluene (1120 mL) was added slowly (over 5 minutes) to the mixture while maintaining the reaction mixture temperature above 35° C. At this point, the total volume was ≈2400 mL. The mixture was well stirred for 10 minutes and then the layers were allowed to settle. A liquid-liquid-liquid triphasic mixture was obtained. The lower layer was removed.

More THF (275 mL) was added to the liquid-liquid biphasic system (total volume 2500 mL). Upon stirring, a clear, single liquid layer appeared. The mixture of THF, water and toluene was next distilled under vacuum. The reaction mixture was reduced from ≈2500 mL to ≈1650 mL under 170-145 τ at 35-38° C. in the vessel (jacket temperature: 47.5° C.). During this operation, the reaction mixture became cloudy and thick. When the volume was ≈2100 mL, the reaction mixture turned into a slurry. Once the volume was ≈1650 mL, distillation was stopped.

Upon overnight cooling under agitation, a thick slurry formed. The slurry was filtered over filter paper (Whatman No 3, 140 mm diameter). The wet cake was then reslurried on the Buchner filter several times in MTBE (high purity) in order to eliminate Z-(D)BIP. Although washing with MTBE may be used, reslurrying in MTBE was surprisingly more effective.

The wet cake (111 g) was placed in the oven (150 T. room temperature, nitrogen flow) and dried for 3.5 days. 98.1 g of Z-(D)BIP-(D)Arg-NH₂.HCl was obtained.

The combined product (126 g) was stirred for 26 hours in the presence of MTBE (1900 mL, 15 volumes) in the 3 L jacketed vessel. The product was filtered over filter paper Whatman No 3 (140 mm diameter).

The wet cake was slurried again in 1900 mL MTBE and stirred for 16 hours in the 3 L jacketed vessel. Upon filtration over filter paper (Whatman No 3, 140 mm diameter) and drying of the wet cake (127.3 g, 150 r, room temperature, nitrogen flow, 20 hours), 121.7 g of Z-(D)BIP-(D)Arg-NH₂.HCl was obtained. This corresponds to 76.7% isolated yield (uncorrected for purity).

### Stage 2: Z-Deprotection (hydrogenolysis)

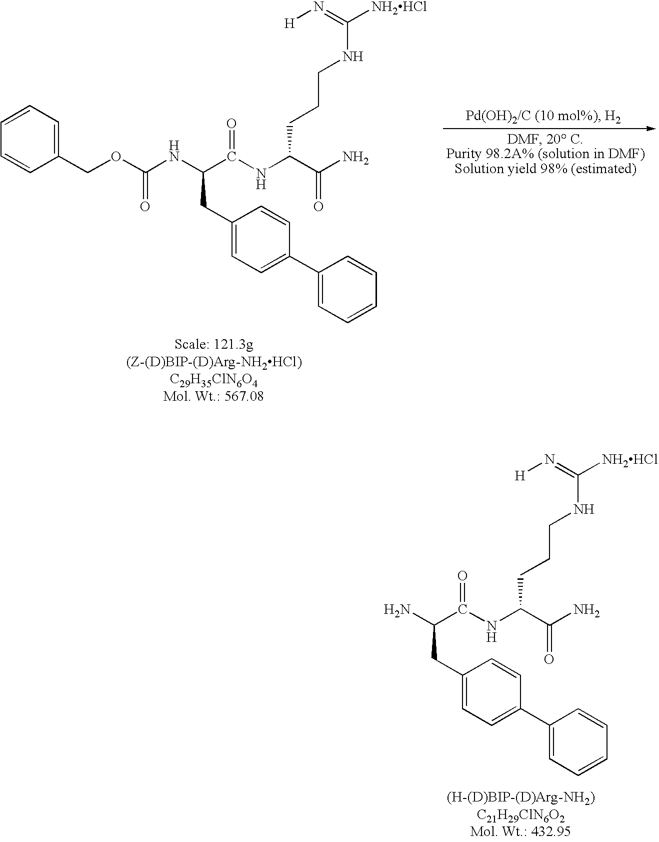

Z-(D)BIP-(D)Arg-NH₂.HCl (121.3 g) was hydrogenolyzed upon treatment with Pd(OH)₂ (20 wt. %, 60% moisture, Aldrich, 28.5 g, 10 mol %) and H₂ (Aldrich) at atmospheric pressure (sub-surface sparging) and room temperature in DMF (Fisher A.C.S., 485 mL).

After 5 hr30 min, the catalyst was removed by filtration through Celite (Celite 521, Aldrich, 38 g), to give a dark colored solution of H-(D)BIP-(D)Arg-NH₂.HCl. in DMF.

### Stage 3: Coualing of H-(D)BIP-(D)Arg-NH2.2HCl with Z-(D)Glu(OBn)-OH

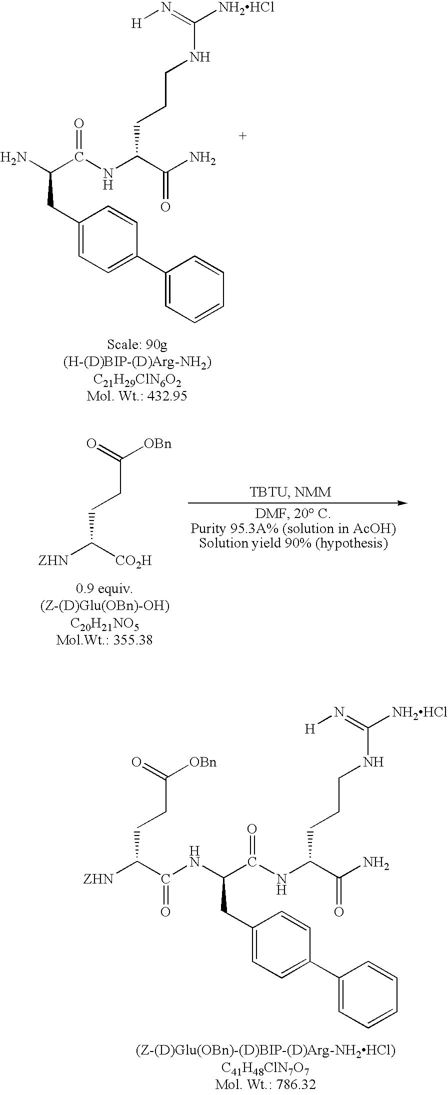

The TBTU-activated Z-(D)Glu(OBn)-OH was prepared in a separate vessel by slowly (over 22 minutes) adding NMM (Sigma-Aldrich 99%, 22.4 mL, 0.97 equiv.) to a solution of Z-(D)Glu(OBn)-OH (Bachem, 65.0 g, 0.87 equiv.) and TBTU (Novabiochem, 60.1 g, 0.90 equiv.) in DMF (Fisher A.C.S., 325 mL) at 235 1° C. The reaction was mildly exothermic: the temperature rose up to 26±1° C.

Thirty minutes after the end of the addition, this solution was added dropwise (over 25 minutes) to a mixture containing H-(D)BIP-(D)Arg-NH₂.HCl. in DMF obtained in Stage 2 (estimated 90.7 g), in solution in DMF (690 mL), and NMM (Sigma-Aldrich 99%,22.4 mL, 0.97 equiv., pH ≈9).

After 2 hours the dark colored homogeneous reaction mixture was divided into 4 portions of 288 mL each. Each portion was diluted with ethyl acetate (bulk, 1520 mL) and de-ionized water (1520 mL) in a 3 L jacketed vessel. To prevent transformation of the mixture into a gel, the solution temperature is preferably maintained above 40° C. (45° C. in the jacket). The content was stirred for about 10 minutes and allowed to settle for another 10 minutes. The aqueous layer (≈7.3 L) was discarded and the organic phases were combined and filtered over Celite. The filtrate was yellow-colored.

Acetic acid (Fisher, A.C.S., 1225 mL) was added to the organic filtrate to prevent its transformation into a gel. The resulting solution was concentrated (170→25 τ, 45-47° C. bath temperature) until a target volume of 620 mL (684 g) was reached. The resulting solution containing Z-(D)Glu(OBn)-(D)BIP-(D)Arg-NH₂.HCl in acetic acid was yellow-colored and slightly cloudy (small amount of solid in suspension).

### Stage 4: Z-Deprotection (hydrogenolysis)

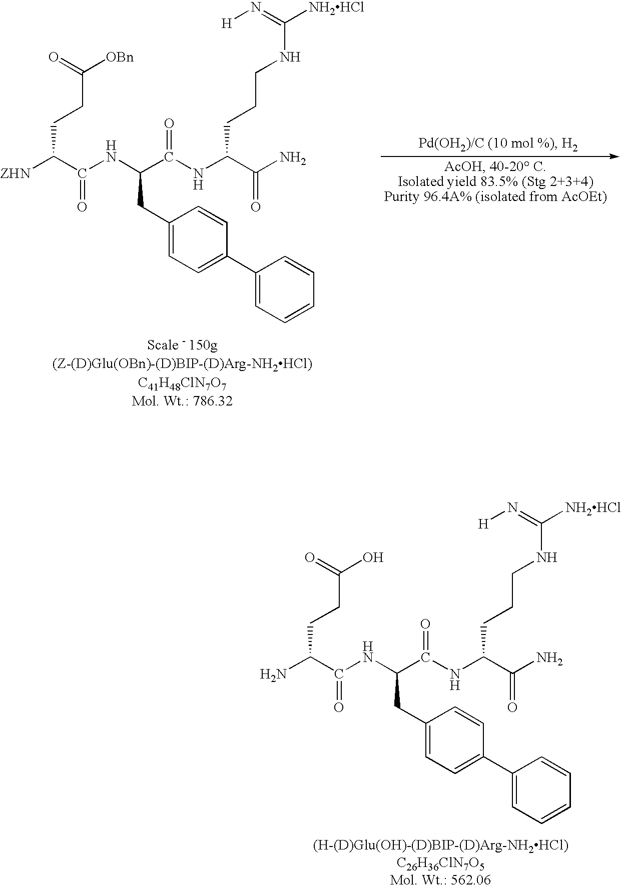

The solution of Z-(D)Glu(OBn)-(D)BIP-(D)Arg-NH₂.HCl in acetic acid obtained in Stage 3 (estimated 148.3 g in 730 mL acetic acid) was heated to 35-40° C. in a water bath to transform it from a gel into an easily stirrable solution. This solution was transferred to a 3 L jacketed vessel (jacket 40° C.).

The solution was hydrogenolyzed upon treatment with Pd(OH)₂ (20 wt. %, 60% moisture, Aldrich, 20.5 g, 8.2 mol %) and H₂ (Aldrich) at atmospheric pressure (sub-surface sparging).

Once sparging had begun, the jacket heater was turned off and the temperature slowly went back to 25° C.

After 22 h, the reaction. mixture was filtered through Celite (Celite 521, Aldrich, 27 g) and the Celite washed with acetic acid.

Half of the filtrate (430 mL) was added dropwise (over 16 minutes) to ethyl acetate (bulk, 1950 mL,) under vigorous agitation. The slurry was filtered over filter Whatman No 1 (150 mm diameter). This operation was repeated on the second half of the batch and the slurry was filtered over the same filter. The wet cake was reslurried 5 times in ethyl acetate (250 mL) to give a powdery solid. The wet cake (448 g) was next dried in a vacuum oven (150 τ, room temperature, nitrogen flow, 6 days).

100.4 g of H-(D)Glu-(D)BIP-(D)Arg-NH₂.HCl was isolated, which corresponds to a 83.5% isolated yield (stage 2+3+4, uncorrected for purity).

### Stage 5: Acetylation

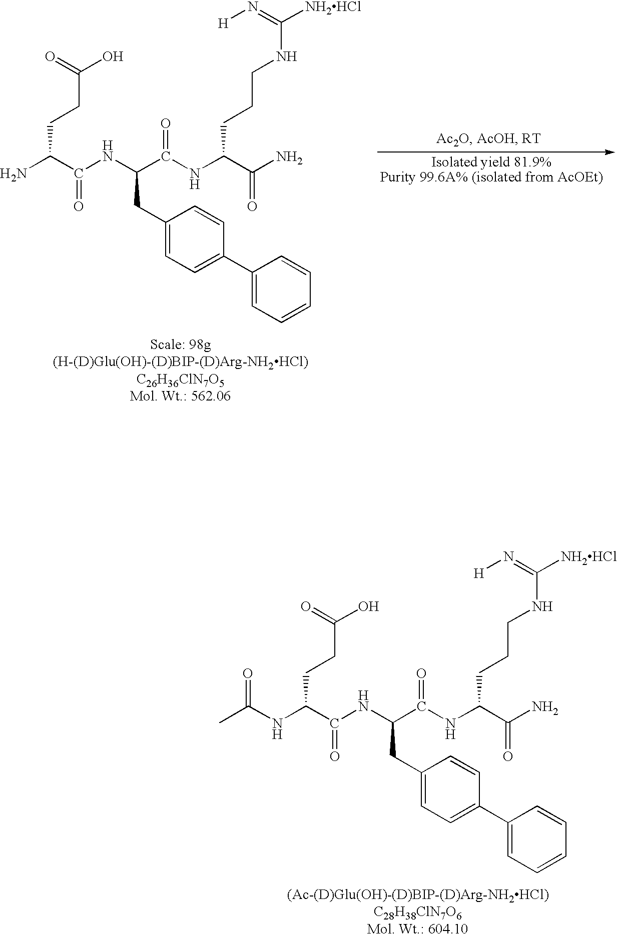

H-(D)Glu-(D)BIP-(D)Arg-NH₂.HCl (98.1 g) was dissolved in acetic acid (Fisher, A.C.S., 980 mL), treated with acetic anhydride (Aldrich 99.5%, 44.5 g, 2.45 equiv.) and stirred at 20° C. for 3 hours 40 minutes.

The reaction mixture (a 1 L) was added dropwise (over 1 hour) to ethyl acetate (bulk, 4.9 L) under vigorous agitation. Half an hour after the end of the addition, the slurry was filtered over 2 separate filters (Whatman No 1, 150 mm diameter). The product was left on the filters, under vacuum, for 1.5 day.

The solid was ground in a mortar and dried in the oven (150 τ, room temperature, nitrogen flow, 1 day).

86.4 g of Ac-(D)Glu-(D)BIP-(D)Arg-NH₂.HCl was isolated, which corresponds to an 81.9% isolated yield (uncorrected for purity).

### Example 2

### Synthesis of Certain Compounds

### Stage 1: Coupling of Z-(D)BIP with H-(D)Arg-NH2.2HCl

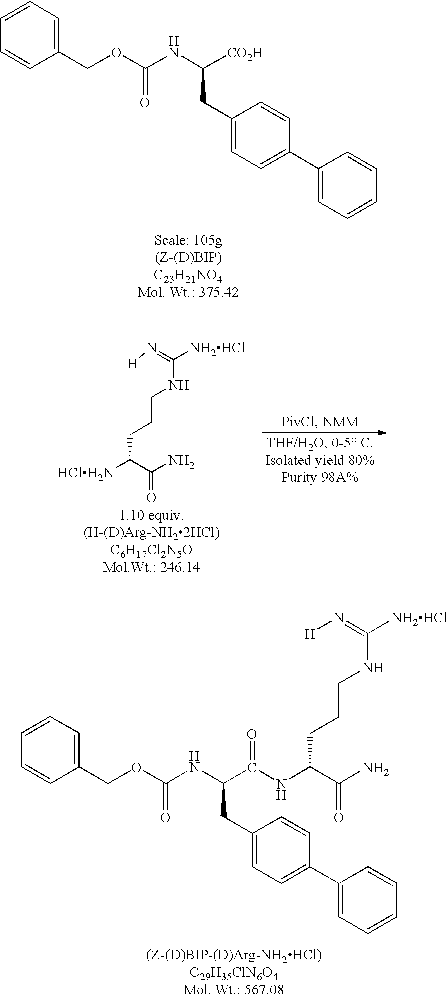

The mixed anhydride Z-(D)BIP-OPiv was prepared by slowly adding N-methylmorpholine (NMM) (Sigma-Aldrich 99%, 29.4 g, 1.04 eq.) to a chilled (<5° C.) mixture of Z-(D)BIP (Synthetec, 105 g) and pivaloyl chloride (PivCl) (Aldrich 99%, 35.4 g, 1.05 eq.) in THF (Sigma-Aldrich 99+%, 630 mL, 6 volumes vs Z-(D)BIP). The reaction mixture was stirred for an additional 30 minutes at <5° C.

The cold (<5° C.) solution of mixed anhydride Z-(D)BIP-OPiv was slowly added to a cold solution of H-(D)Arg-NH₂.2HCl (Bachem, 75.7 g, 1.1 eq.) in deionized water (303 mL) and NMM (Sigma-Aldrich 99%, 31.1 g, 1.10 eq.). The reaction mixture temperature was maintained below 5° C. The reaction was stirred at <5° C. overnight.

The liquid-liquid biphasic reaction mixture was allowed to warm-up to room temperature and the organic phase was separated.

2.5 volumes of brine and 0.225 volumes of N-methylmorpholine were charged to the top organic layer and allowed to stir for 10 minutes. The layers were separated, and the aqueous layer was retained for HPLC analysis (pH ˜8.5).

2.5 volumes of brine and 0.375 volumes of 18% aq. HCl were charged to the organic layer. The solution was allowed to stir for 10 minutes, and the aqueous layer was separated and retained for HPLC analysis (pH ˜2 -4).

1.25 volumes of brine was added to organic layer for last wash and mixture stirred for 10 minutes. The layers were separated, and the aqueous layer was discarded (pH ˜6).

2.5 volumes of Tetrahydrofuran (THF) was added to the organic layer followed by 10 volumes of Methyl-tert butyl ether (MTBE). Solvent exchange from MTBE/THF to MTBE was preformed by vacuum distillation at 25-30° C. to about 40% reduction.

About 7 volumes MTBE was charged to the reactor making up to the starting volume. Vacuum distillation was repeated with stirring to remove any left over THF and water. Stage-1 product crystallized out.

The product was filtered and the cake was washed with 5 volumes of MTBE.

Re-slurry of isolated product in 10 volumes of MTBE for 1 hour typically yielded desirable product of about 99% purity. The pure product was dried under vacuum at room temperature for 18-24 hours. Yield was ˜80% and ˜98 A % pure. The product was a white free flowing powdery solid.

### Stage 2: Z-Deprotection (hydrogenolysis)

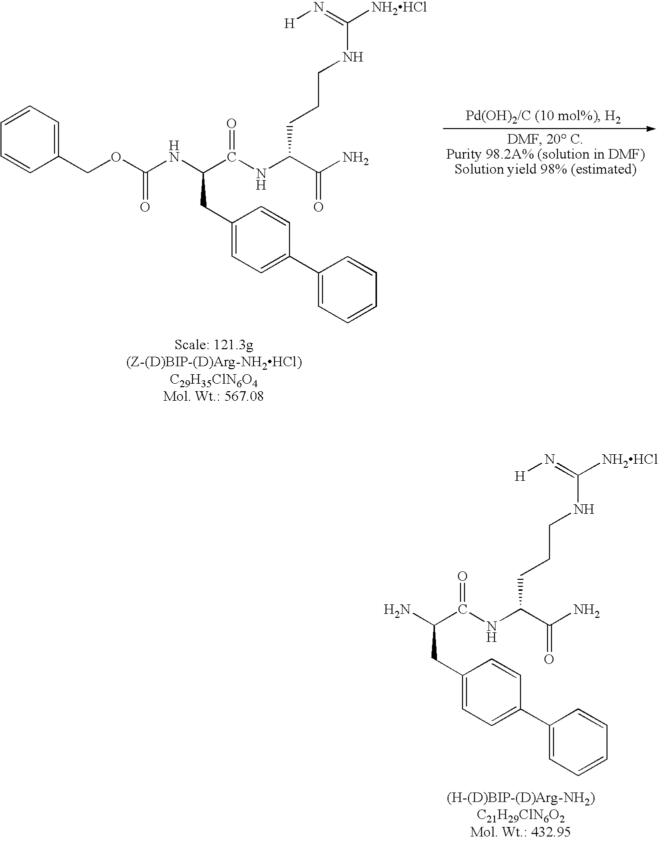

Z-(D)BIP-(D)Arg-NH₂.HCl (121.3 g) was hydrogenolyzed upon treatment with Pd(OH)₂ (20 wt. %, 60% moisture, Aldrich, 28.5 g, 10 mol %) and H₂ (Aldrich) at atmospheric pressure (sub-surface sparging) and room temperature in DMF (Fisher A.C.S., 485 mL).

After 5 hour 30 minutes, the catalyst was removed by filtration through Celite (Celite 521, Aldrich, 38 g), to give a dark colored solution of H-(D)BIP-(D)Arg-NH₂.HCl in DMF.

### Stage 3: Coupling of H-(D)BIP-(D)Arj-NH2.2HCl with Z-(D)Glu(OBn)-OH

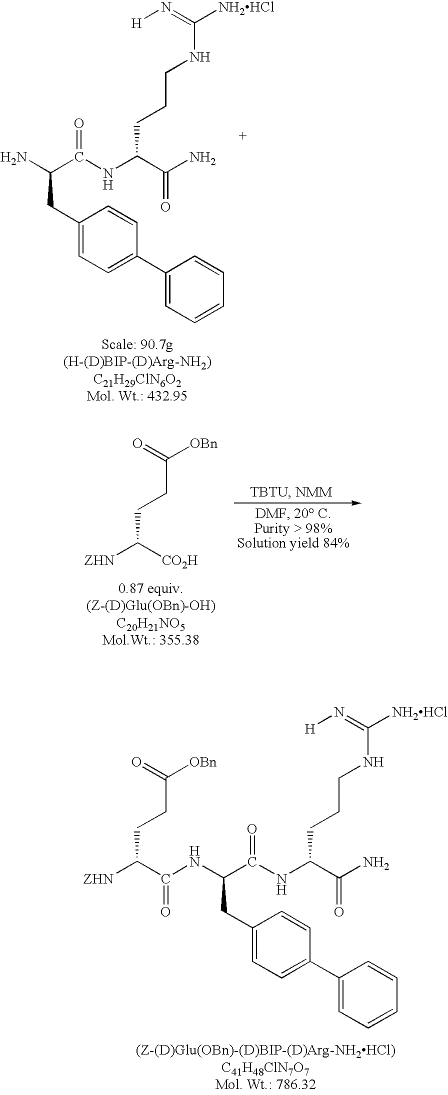

The TBTU-activated Z-(D)Glu(OBn)-OH was prepared in a separate vessel by slowly adding NMM (Sigma-Aldrich 99%, 22.4 mL, 0.97 equiv.) to a solution of Z-(D)Glu(OBn)-OH (Bachem, 65.0 g, 0.87 equiv.) and TBTU (Novabiochem, 60.1 g, 0.90 equiv.) in DMF (Fisher A.C.S., 325 mL) at 20±1° C.

Thirty minutes after the end of the addition, this solution was added dropwise to a mixture containing H-(D)BIP-(D)Arg-NH₂.HCl. in DMF obtained in Stage 2 (estimated 90.7 g), in solution in DMF (690 mL), and NMM (Sigma-Aldrich 99%, 22.4 mL, 0.97 equiv., pH ≈9).

The reaction solution was warmed to 40° C. Ethyl Acetate (4.2 volumes vs. the volume of the reaction mixture) was added to the reaction mixture followed by 4.2 volumes of 40° C. deionized water. The mixture was stirred for 10 minutes and the layers separated. The solution was maintained at 40° C.

Optionally, the bottom aqueous layer was extracted with 1 volume of Ethyl Acetate (40° C.), and organic layers were combined. Aqueous NMM was added to the organic layer, and the phases split. Saturated NaCl solution (brine), 0.5 volume, was added to the organic layer and stirred. The product precipitated out from the solution, and was filtered to remove the water and Ethyl Acetate. The filter cake was washed the with 2 volumes of Ethyl Acetate. The product was dried under vacuum at room temperature for 18-24 hours. Yield was 84% with a purity of >98 % by HPLC.

### Stage 4: Z-Deprotection (hydrogenolysis)

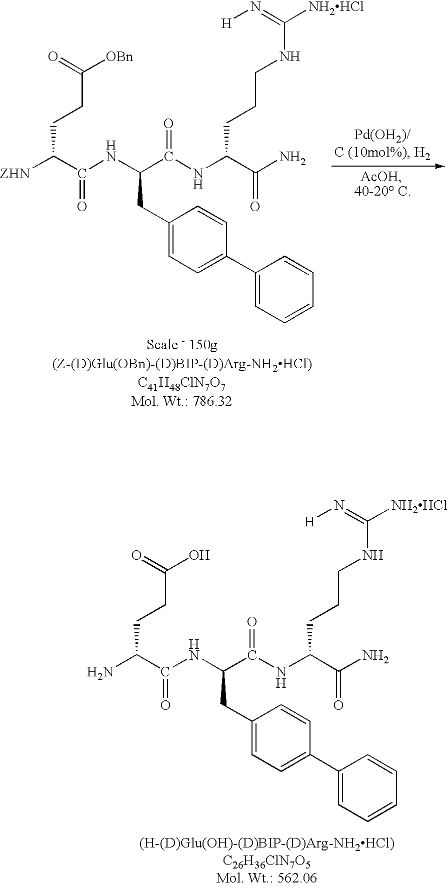

Stage -3 product and acetic acid (7 volumes) were charged in a hydrogenation reactor. The mixture was stirred at 40-50° C. under N₂ to obtain a clear to slightly opaque solution. The resulting solution was cooled to about 20-25° C. 20% Pd(OH)₂/C (60% moisture) was charged at 10 mol % vs. the Stage -3 product. N₂ was replaced with H₂ by empting the N₂ and adding 50psi of H₂ then evacuating the reactor and recharging 50psi of hydrogen gas. This mixture was stirred for about 25-30 hours at room temperature. After the reaction was complete the reactor was evacuated and the mixture was filtered over a combination of fiberglass -Whatman 3 filter papers. The reactor was washed with minimal amount of Acetic acid and filtered and the filtrate was combined with the reaction mixture. The catalyst was washed with 0.5 volume of acetic acid. The solution was a clear orange-yellow liquid. This was used directly for Stage-5 acetylation reaction assuming 100% yield.

### Stage 5: Acetylation

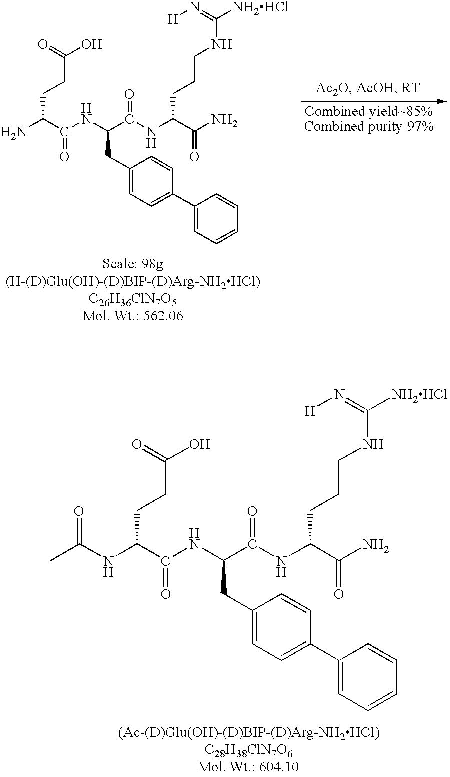

A clear 225 mL acetic acid solution from Stage-4 containing estimated 20 g, 35.6 mmol of Stage-4 product was charged in 500 mL 3-neck round bottom flask equipped with a nitrogen gas inlet. Acetic anhydride, 22 g, 215.7 mmol, 6.1 eq, was added to the acetic solution. The reaction mixture was stirred at room temperature for 5 hours.

The reaction mixture was allowed to stir for additional 6-8 hours for crystallization/precipitation of the product. The white slurry was cooled to 8-10° C. to complete the crystallization/precipitation. The white product was filtered. The product cake was subjected to prolonged vacuum suction. The filter cake was washed twice with 25 mL ethyl acetate by soaking and then suctioning the filter cake (2×25 mL).

The product was dried at room temperature under vacuum for 24 hours. Product yield was 12 g (˜60%) with a purity of about 98.7%.

Mother liquor was diluted with about 900 mL ethyl acetate (3 volumes of acetic acid ) and stirred for 10 minutes. White precipitate was filtered and washed with ethyl acetate. Product was dried. Yield of 2^{nd} crop was about 6.5 g.

Combined total yield of Stage 5 product was 85% with a combined purity of about 97%

Recrystallization of final Product (Stage-5)

15 g of dry crude Stage-5 product was taken into a 500 mL flask. 150 mL (10 volumes) acetic acid was added to the flask and the slurry was heated to 100-110° C. with stirring. A clear to cloudy solution was obtained after 15-30 min. stirring (Solution A).

Solution A was allowed to cool to room temperature with occasional stirring over 2 hours. A small amount of solids appeared which were filtered off.

To the clear solution, 150 mL ethyl acetate (10 volumes) was added with stirring. White product crystallized out. Another 150 mL ethyl acetate was added to the slurry to ensure complete crystallization. The slurry was allowed stand for 15 minutes.

The white solids were filtered. The wet product cake was washed twice with 100 mL EtOAc each. The product was transferred to a dish and dried at room temperature under vacuum for about 20 hours.

Dry product yield was 14.4 g (96% recovery) and free flowing. Purity of product was better than 99%.

In a separate experiment, Solution A was allowed to cool to ambient temperature over several hours and the product was filtered to give a white solid. The white solid was washed with ethyl acetate, and dried under vacuum. Dried product was obtained in 80% yield. Optionally, the product may be recrystallized from acetic acid.

Many modifications and variations of the embodiments described herein may be made without departing from the scope, as is apparent to those skilled in the art. The specific embodiments described herein are offered by way of example only.

What is claimed is:

1. A method of preparing a compound having the formula: 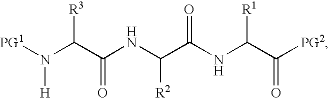
or a pharmaceutically acceptable salt thereof;
wherein R¹ is a basic side chain and R³ is an acidic side chain or R¹ is an acidic side chain and R³ is a basic side chain;
R¹ and R³ can be protected or unprotected side chain;
R² is a hydrophobic side chain;
PG¹ and PG² are protecting or capping groups;
the method comprising coupling a compound of the formula: 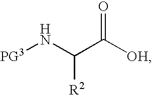
wherein PG³ is a protecting group, and a compound of the formula: 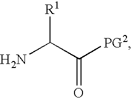
or a mineral acid salt or organic acid salt thereof, thereby forming a compound having the formula: 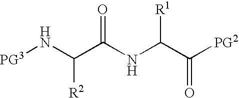
or a mineral acid salt or organic acid salt thereof;
deprotecting the compound of the formula: 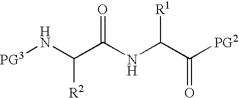
or a mineral acid salt or organic acid salt thereof by removing PG³, thereby forming a compound having the formula: 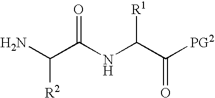
or a mineral acid salt or organic acid salt thereof; coupling the compound of the formula: 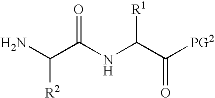
or a mineral acid salt or organic acid salt thereof and a compound of the formula: 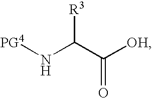
wherein PG⁴ is a protecting group, thereby forming a compound of the formula: 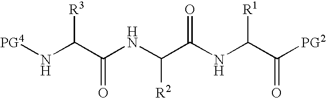
or a mineral acid salt or organic acid salt thereof;
deprotecting the compound of the formula: 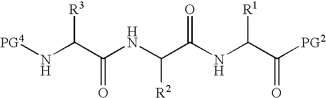
or a mineral acid salt or organic acid salt thereof
by removing PG⁴, thereby forming a compound having the formula: 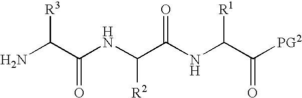
or a mineral acid salt or organic acid salt thereof; and
protecting the compound of the formula: 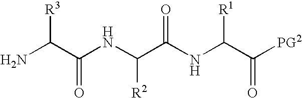
at the terminal amino end, thereby forming a compound having the formula: 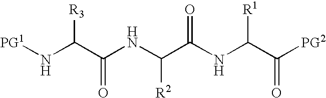
or a mineral acid salt or organic acid salt thereof,
optionally converting the mineral acid salt or organic salt to a pharmaceutically acceptable salt.

2. The method of claim 1, further comprising removing PG¹ and/or PG².

3. The method of claim 1, wherein when the compound is obtained in a free (non-salt) form, further comprising converting the compound to a pharmaceutically acceptable salt form.

4. The method of claim 1, wherein when the compound is obtained in a salt form, further comprising converting the compound to a different pharmaceutically acceptable salt form.

5. The method of claim 1, wherein any of the coupling steps is facilitated with a coupling agent.

6. The method of claim 5, wherein the coupling agent is pivaloyl chloride or TBTU.

7. The method of claim 1, wherein PG³ and PG⁴ are benzyloxycarbonyl groups.

8. The method of claim 7, wherein the deprotecting step is performed by hydrogenolysis.

9. The method of claim 8, wherein the deprotecting step is performed with Pd(OH)₂ and hydrogen.

10. The method of claim 1, wherein PG²is an amino group.

11. The method of claim 1, wherein PG¹ comprises a group R^{x}—CO— wherein R^{x} is selected from the group consisting of methyl, phenyl-CH₂—, di-tert-butyl-4-hydroxy-phenyl, naphthyl, substituted naphthyl, 9-fluorenylmethoxy-, biphenyl, substituted phenyl, substituted heterocycles, alkyl, aryl, substituted aryl, cycloalkyl, fused cycloalkyl, saturated heteroaryl, and substituted saturated heteroaryl.

12. The method of claim 11, wherein PG¹ is an acetyl group.

13. The method of claim 12, wherein the acetylation step is performed with acetic anhydride.

14. The method of claim 1, further comprising removing PG¹ of the compound having the formula:

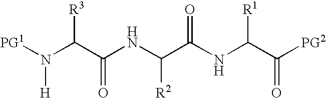

15. The method of claim 1, further comprising removing PG² of the compound having the formula:

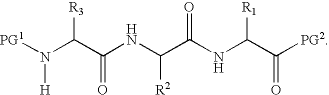

16. The method of claim 1 wherein each amino acid is independently L or D.

17. The method of claim 1, wherein all of the amino acids are L.

18. The method of claim 1, wherein all of the amino acids are D.

19. A method of preparing a compound having the formula: 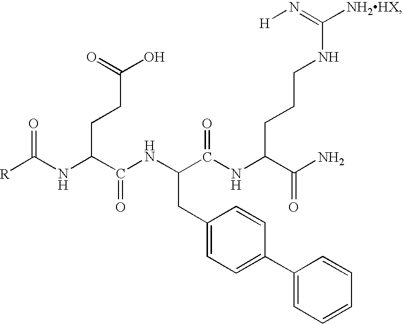
wherein HX is a mineral salt, organic salt or a pharmaceutically acceptable salt; and R is unsubstituted or substituted alkyl or an unsubstituted or substituted aryl; the method comprising coupling a compound of the formula: 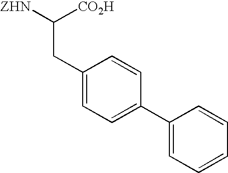
, wherein Z is a benzyloxycarbonyl group, and a compound of the formula: 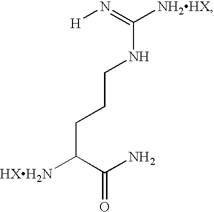
thereby forming a compound of the formula: 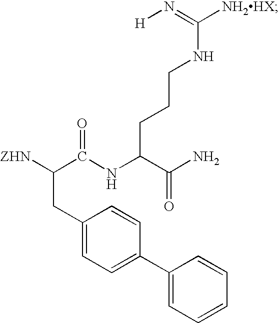
deprotecting the compound of the formula: 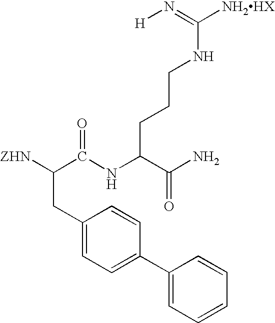
by removing the benzyloxycarbonyl group at the amino end, thereby forming a compound of the formula: 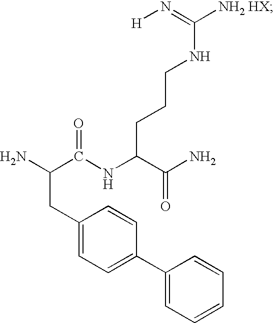
coupling the compound of the formula: 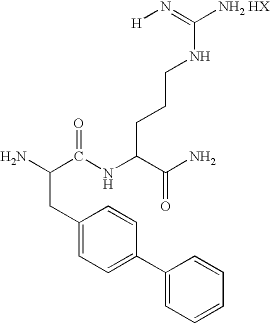
and a compound of the formula: 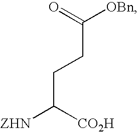
wherein Z is a benzyloxycarbonyl group and Bn is a benzyl group, thereby forming a compound of the formula: 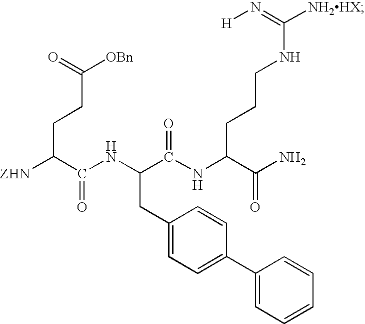
deprotecting the compound of the formula: 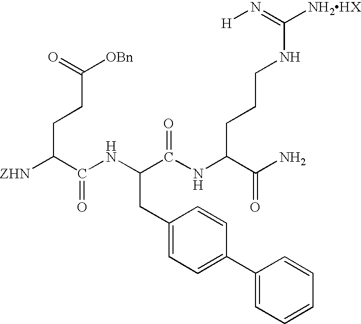
by removing the benzyloxycarbonyl group and the benzyl group, thereby forming a compound of the formula: 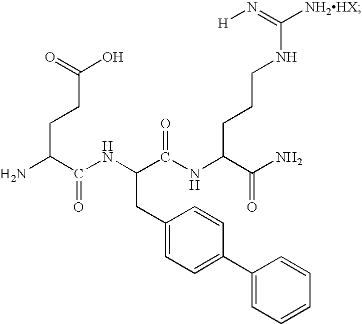
and acylating the compound of the formula: 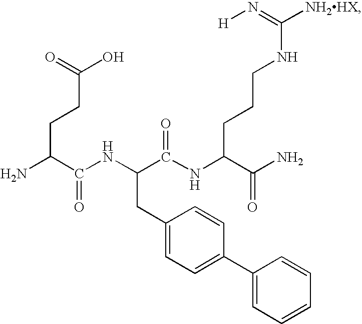
thereby forming a compound of the formula:

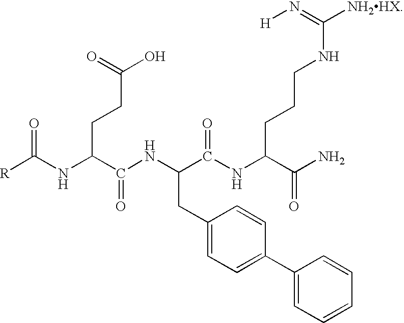

20. The method of claim 19 wherein each amino acid is independently L or D.

21. The method of claim 19, wherein all of the amino acids are L.

22. The method of claim 19, wherein all of the amino acids are D.

23. The method of claim 19, wherein R is unsubstituted alkyl which is —(CH₂)ₙ—CH₃, wherein n is 0-5.

24. The method of claim 19, wherein any of the coupling steps is facilitated with a coupling agent.

25. The method of claim 19, wherein the coupling agent is pivaloyl chloride or TBTU.

26. The method of claim 19, wherein any of the deprotecting steps is performed by hydrogenolysis.

27. The method of claim 26, wherein any of the deprotecting steps is performed with Pd(OH)₂ and hydrogen.

28. The method of claim 19, wherein the acylation step is performed with acetic anhydride.

29. The method of claim 19, wherein an intermediate is isolated by a method comprising the steps of:
washing an organic phase containing the intermediate with a saturated salt solution; and
precipitating the intermediate from the organic phase.

30. The method of claim 29, wherein the precipitation occurs by distilling the organic phase until the intermediate crystallizes out of the organic phase.

31. A compound having the formula: 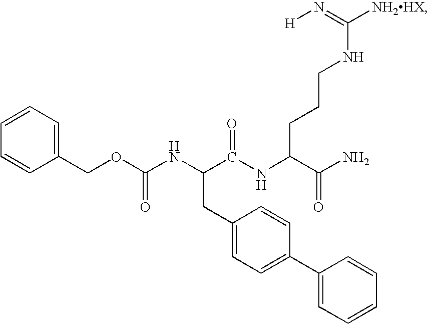
wherein HX is a mineral salt or organic salt.

32. A compound having the formula: 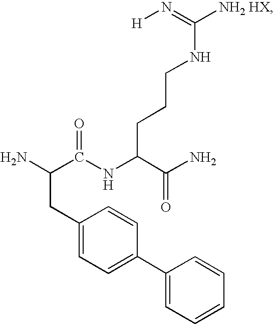
wherein HX is a mineral salt or organic salt.

33. A compound having the formula: 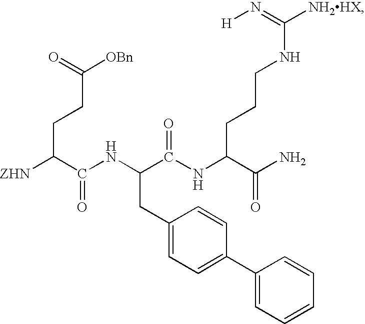
wherein HX is a mineral salt or organic salt.

34. A compound having the formula: 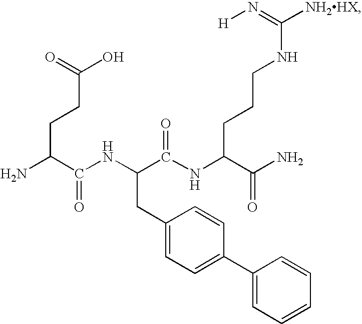
wherein HX is a mineral salt or organic salt.

35. A compound having the formula: 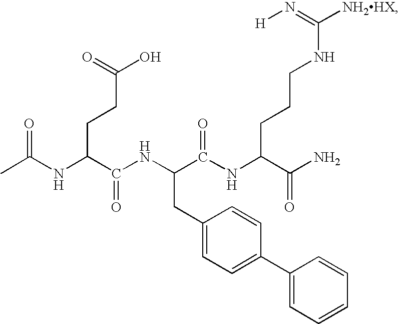
wherein HX is a mineral salt or organic salt.

36. A compound having the formula: 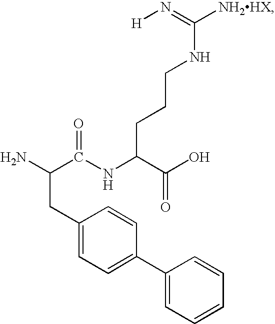
wherein HX is a mineral salt or organic salt.

37. A compound having the formula: 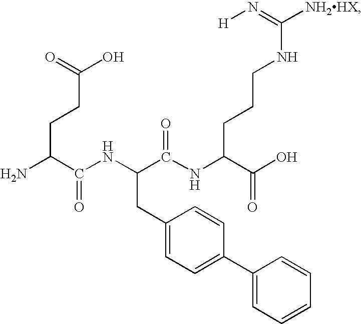
wherein HX is a mineral salt or organic salt.

38. A compound having the formula: 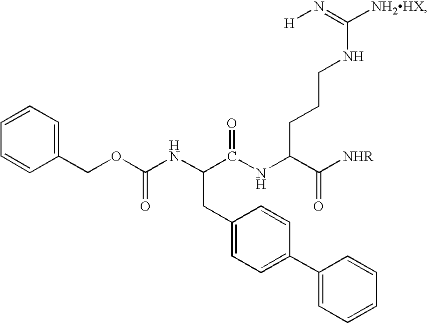
wherein HX is a mineral salt or organic salt; and
wherein R is H or an unsubstituted or substituted alkyl or an unsubstituted or substituted aryl.

39. A compound having the formula: 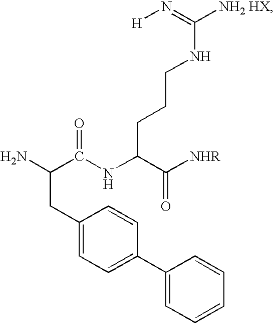
wherein HX is a mineral salt or organic salt; and
wherein R is H or an unsubstituted or substituted alkyl or an unsubstituted or substituted aryl.

40. A compound having the formula: 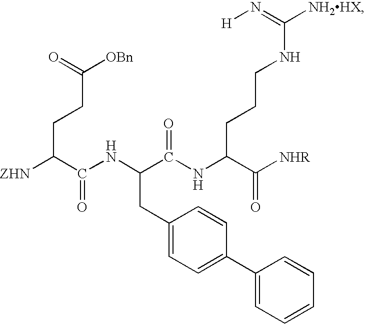
wherein HX is a mineral salt or organic salt; and
wherein R is H or an unsubstituted or substituted alkyl or an unsubstituted or substituted aryl.

41. A compound having the formula: 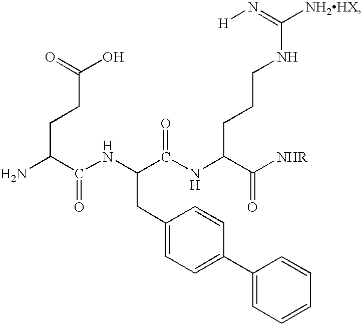
wherein HX is a mineral salt or organic salt; and
wherein R is H or an unsubstituted or substituted alkyl or an unsubstituted or substituted aryl.

42. A compound having the formula: 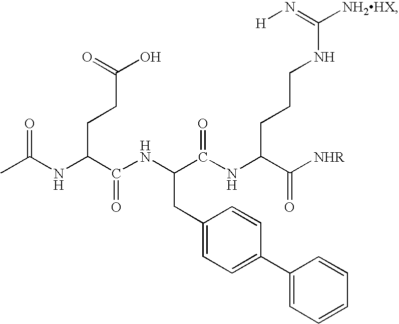
wherein HX is a mineral salt, organic salt or a pharmaceutically acceptable salt; and
wherein R is H or an unsubstituted or substituted alkyl or an unsubstituted or substituted aryl.

43. A compound produced by the method of claim 1 or 19.
